SEQUENCE LISTING (34 sequences)

SEQ ID NO: 1 - DNA, 26 bp, Artificial Sequence
  Description of Artificial Sequence Synthetic primer
aaggaagaggagactctgcgcagagc

SEQ ID NO: 2 - DNA, 34 bp, Artificial Sequence
  Description of Artificial Sequence Synthetic primer
taaatgtatgtatgtgggtgggtgtgtctacagg

SEQ ID NO: 3 - DNA, 20 bp, Artificial Sequence
  Description of Artificial Sequence Synthetic primer
acttccccaaatcactgtgg

SEQ ID NO: 4 - DNA, 20 bp, Artificial Sequence
  Description of Artificial Sequence Synthetic primer
gtcactcactttgcccctgt

SEQ ID NO: 5 - DNA, 25 bp, Artificial Sequence
  Description of Artificial Sequence Synthetic primer
gcttgtagtaattgttcataagtgg

SEQ ID NO: 6 - DNA, 23 bp, Artificial Sequence
  Description of Artificial Sequence Synthetic primer
gagcgtatgtctactatacgcca

SEQ ID NO: 7 - DNA, 20 bp, Artificial Sequence
  Description of Artificial Sequence Synthetic primer
agctttggttggttttggtg

SEQ ID NO: 8 - DNA, 20 bp, Artificial Sequence
  Description of Artificial Sequence Synthetic primer
cctggaaacaaaaggcattc

SEQ ID NO: 9 - DNA, 24 bp, Artificial Sequence
  Description of Artificial Sequence Synthetic primer
aagaggctgagtcagaaggattgg

SEQ ID NO: 10 - DNA, 27 bp, Artificial Sequence
  Description of Artificial Sequence Synthetic primer
gcaacatcattttaatatcctgcacag

SEQ ID NO: 11 - DNA, 22 bp, Artificial Sequence
  Description of Artificial Sequence Synthetic primer
ttgttctaacacctgccactct

SEQ ID NO: 12 - DNA, 19 bp, Artificial Sequence
  Description of Artificial Sequence Synthetic primer
ggcaaacctgagtctcaca

SEQ ID NO: 13 - DNA, 20 bp, Artificial Sequence
  Description of Artificial Sequence Synthetic primer
ctcatgaggacccaggtgat

SEQ ID NO: 14 - DNA, 20 bp, Artificial Sequence
  Description of Artificial Sequence Synthetic primer
ggttgaggcagctatggaga

SEQ ID NO: 15 - DNA, 20 bp, Artificial Sequence
  Description of Artificial Sequence Synthetic primer
catctttgctgtcgtcctca

SEQ ID NO: 16 - DNA, 20 bp, Artificial Sequence
  Description of Artificial Sequence Synthetic primer
ctgtgaaggatgcccagaat

SEQ ID NO: 17 - DNA, 20 bp, Artificial Sequence
  Description of Artificial Sequence Synthetic primer
catttgcaaacagaggctca

SEQ ID NO: 18 - DNA, 20 bp, Artificial Sequence
  Description of Artificial Sequence Synthetic primer
tgtgacagagcaaggcaaag

SEQ ID NO: 19 - DNA, 20 bp, Artificial Sequence
  Description of Artificial Sequence Synthetic primer
tgctgtgacccactctgtct

SEQ ID NO: 20 - DNA, 20 bp, Artificial Sequence
  Description of Artificial Sequence Synthetic primer
ccagaaggttgcacttgtcc

SEQ ID NO: 21 - DNA, 20 bp, Artificial Sequence
  Description of Artificial Sequence Synthetic primer
accccagggctctatgggaa

SEQ ID NO: 22 - DNA, 20 bp, Artificial Sequence
  Description of Artificial Sequence Synthetic primer
tgagggcacaagaagcccct

SEQ ID NO: 23 - DNA, 20 bp, Artificial Sequence
  Description of Artificial Sequence Synthetic primer
cccaatggatgatgacttcc

SEQ ID NO: 24 - DNA, 21 bp, Artificial Sequence
  Description of Artificial Sequence Synthetic primer
agtggtggcattagcagtagg

SEQ ID NO: 25 - DNA, 20 bp, Artificial Sequence
  Description of Artificial Sequence Synthetic primer
acaggcagggtggtgtatgt

SEQ ID NO: 26 - DNA, 20 bp, Artificial Sequence
  Description of Artificial Sequence Synthetic primer
cacctgtcagggcattttct

SEQ ID NO: 27 - DNA, 26 bp, Artificial Sequence
  Description of Artificial Sequence Synthetic primer
gtttgaaattttaaagtacttttgat

SEQ ID NO: 28 - DNA, 22 bp, Artificial Sequence
  Description of Artificial Sequence Synthetic primer
tttcaaattattgtttcattgc

SEQ ID NO: 29 - DNA, 46 bp, Artificial Sequence
  Description of Artificial Sequence Synthetic oligonucleotide
cacacacacacacacacacacacacacacacacacacacacacaca

SEQ ID NO: 30 - DNA, 76 bp, Artificial Sequence
  Description of Artificial Sequence Synthetic oligonucleotide
cacacacacacacacacacacacacacacacacacacacacacacacacacacacacacacacacacacacacaca

SEQ ID NO: 31 - DNA, 56 bp, Artificial Sequence
  Description of Artificial Sequence Synthetic oligonucleotide
cacacacacacacacacacacacacacacacacacacacacacacacacacacaca

SEQ ID NO: 32 - DNA, 16 bp, Artificial Sequence
  Description of Artificial Sequence Synthetic oligonucleotide
cacacacacacacaca

SEQ ID NO: 33 - DNA, 48 bp, Artificial Sequence
  Description of Artificial Sequence Synthetic oligonucleotide
cacacacacacacacacacacacacacacacacacacacacacacaca

SEQ ID NO: 34 - DNA, 74 bp, Artificial Sequence
  Description of Artificial Sequence Synthetic oligonucleotide
cacacacacacacacacacacacacacacacacacacacacacacacacacacacacacacacacacacacaca

(19) United States
(12) Patent Application Publication
(10) Pub. No.: US 2010/0184773 A1
(43) Pub. Date: Jul. 22, 2010

(54) Germline Polymorphisms in the Angiogenic Pathway Predict Tumor Recurrence in Cancer Therapy

(76) Inventor: Heinz-Josef Lenz, Los Angeles, CA (US)

Correspondence Address: FOLEY & LARDNER LLP, 975 PAGE MILL ROAD, PALO ALTO, CA, 94304, US

(21) Appl. No.: 12/600,458

(22) PCT Filed: May 16, 2008
(86) PCT No.: PCT/US08/63898

§ 371 Date: Mar. 25, 2010

Related U.S. Application Data
(60) Provisional application No. 60/939,021, filed on May 18, 2007.

(51) Int. Cl. A61K 31/495; C12Q 1/68; A61K 31/505; A61K 31/513; C40B 40/06

(52) U.S. Cl. 514/249; 435/6; 514/256; 514/274; 506/16

(57) ABSTRACT

The invention provides compositions and methods for determining the likelihood of successful treatment with pyrimidine based antimetabolites and platinum-based alkylating agents. The methods comprise determining the identity of one or more genomic polymorphism present in a predetermined region of a gene of interest and correlating the polymorphism to the predictive response and treatment options. Patients identified as responsive are then treated with the appropriate therapy.

### CROSS-REFERENCE TO RELATED APPLICATION

This application claims the benefit under 35 U.S.C. §119(e) of provisional application U.S. Ser. No. 60/939,021, filed on May 18, 2007, the contents of which is incorporated by reference into the present disclosure in its entirety.

### STATEMENT AS TO FEDERALLY SPONSORED RESEARCH

This invention was made with Government support under NIH Grant 5, P30CA14089-271. Accordingly, the Government may have rights in this invention.

### FIELD OF THE INVENTION

This invention relates to the field of pharmacogenomics and specifically to the application of genetic polymorphism(s) to diagnose and treat diseases.

### BACKGROUND OF THE INVENTION

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidiscinplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response. For a review of the use of germline polymorphisms in clinical oncology, see Lenz (2004) Clin. Oncol. 22(13):2519-2521; Park et al. (2006) Curr. Opin. Pharma. 6(4):337-344; Zhang et al. (2006) Pharma. and Genomics 16(7):475-483 and U.S. Patent Publ. No. 2006/0115827. For a review of pharmacogenetic and pharmacogenomics in therapeutic antibody development for the treatment of cancer, see Yan and Beckman (2005) Biotechniques 39:565-568 and Lenz, H.-J., Pharmacogenomics and Colorectal Cancer, Chpt. 18 in TRENDS IN CANCER FOR THE 21^{st} CENTURY, 2^{nd} ED., Springer (2006).

The Food and Drug Administration has approved the use of Cetuximab, an antibody to the epidermal growth factor receptor (EGFR), either alone or in combination with Irinotecan (also known as CPT-11 or Camptosar®) to treat patients with EGFR-expressing, metastatic CRC, who are either refractory or intolerant to Irinotecan-based chemotherapy. One recent study (Zhang et al. (2006) Pharmocogenetics and Genomics 16:475-483) investigated whether polymorphisms in genes of the EGFR signaling pathway are associated with clinical outcome in CRC patients treated with single-agent Cetuximab. The study also reported that alleles for VEGF and VEGFR2 (receptor 2) as well as the cyclin D1 (CCND1) A870G and the EGF A61G polymorphisms may be useful molecular markers for predicting clinical outcome in CRC patients in stage II or III CRC. Thus, polymorphic analysis is recognized as a means to identify patients more or less likely to respond to a treatment regimen.

Colorectal cancer is the third most common cancer in the United States. In the year 2007, an estimated 153,000 new cases will be diagnosed and 52,000 people will die from this disease (Jemal. A. et al. (2007) CA Cancer J Clin. 57:43-66). For patients who undergo successful surgery for colon cancer, additional chemotherapy is recommended in stage III disease. 5-Adjuvant chemotherapy with FOLFOX reduces the relative rate of recurrence by 23% and the overall death rate by 31% and is the standard of care for Stage III colon cancer patients (Andre T, et al. (2004) N Engl. J. Med. 350:2343-51; Kuebler, J. P., et al. (2007) J. Clin. Oncol. 25:2198-204; and Moertel, C. G. et al. (1995) Ann. Intern. Med. 122:321-6). Despite advances in the treatment of colorectal cancer, the five year survival rate for metastatic colon cancer is still low, with a median survival of 18-21 months (Douglass et al. (1986) N. Eng. J. Med. 315:1294-1295). Factor influencing prognosis include the stage of the cancer at diagnosis, age and gender of the patient, and as shown herein, the patient's genotype.

Tumor recurrence after curative resection also continues to be a significant problem in the management of colon cancer. Tumor angiogenesis plays an important role in tumor development, tumor progression and metastases (Folkman J. (1971) N. Engl. J. Med. 285:1182-6 and Folkman, J. (1990) J. Natl. Cancer Inst. 82:4-6). When a tumor grows beyond a size of approximately 2 to 3 mm, it requires new and dedicated vasculature (Folkman, J. (1995) N. Engl. J. Med. 333:1757-63). The switch to the angiogenic phenotype involves a change in the local equilibrium between positive and negative regulators of the growth of microvessels (Dameron, K. M. (1994) Science 265:1582-4). This “angiogenic switch” is considered a hallmark of the malignant process and is required for tumor propagation and progression (Naumov, G. N. et al. (2006) Cell Cycle 5:1779-87). Vascular endothelial growth factor (VEGF) and its receptors VEGFR-1 and VEFGR-2 are critical activators of tumor associated angiogenesis.

Recent research identified interleukin-8 (IL-8) and adrenomedullin (AM) as critical mediators of VEGF independent tumor angiogenesis. Induction of IL-8 preserved the angiogenic response in HIF1-α deficient colon cancer cells, suggesting that IL-8 mediates angiogenesis, independently of VEGF (Mizukami, Y. et al. (2005) Nat. Med. 11:992-7). In the postgenomic era, the possibility of individualized cancer treatment is gaining wider acceptance, and numerous germline polymorphisms in genes involved in the angiogenesis pathway that influence differential enzyme function or expression, have been identified. However, there are only a few clinical and potential molecular markers, which can predict tumor recurrence in stage III colon cancer patients. These include microsatellite instability (MIN) and 18q deletions (Watanabe, T. et al. (2001) N. Engl. J. Med. 344:1196-206). The identification of molecular pathways is critical in understanding the mechanisms of tumor relapse and therefore essential in the development of more effective adjuvant treatment strategies.

Other polymorphisms have been reported to be associated with clinical outcome. Twenty-one (21) polymorphisms in 18 genes involved in the critical pathways of cancer progression (i.e., drug metabolism, tumor microenvironment, cell cycle regulation, and DNA repair) were investigated to determine if they will predict the risk of tumor recurrence in rectal cancer patients treated with chemoradiation (Gordon et al. (2006) Pharmacogenomics 7(1):67-88). This invention has undertaken an analysis to determine if genes involved in the tumor angiogenesis pathway independently predict responsiveness to certain treatment regimens.

### DESCRIPTION OF THE EMBODIMENTS

This invention provides methods to: 1) select the appropriate therapy for patients suffering from a gastrointestinal cancer (“GIC”) or lung cancer; and/or 2) identify patients more likely responsive to a selected therapy and/or 3) identify patients having the greater or alternatively less time to tumor recurrence or overall survival after treatment with a pre-selected therapy. The method requires detecting the identity of at least one allelic variant of a predetermined gene selected from the group identified in Tables 1 and 2, below.

TABLE 1
Predictive Polymorphisms For Tumor Recurrence Following Pyrimidine
Based Antimetabolite And Efficacy Enhancing Agent Chemotherapy.

| | Predictive Genotype | |
|---|---|---|
| Allele (Polymorphism) | (Polymorphism) | Measured Response |
| VEGF (936 C/T) | C/T or T/T | Tumor recurrence in stage |
| | | III colon cancer |
| IL-8 (−251 T/A) | T/T or T/A | Tumor recurrence in stage |
| | | III colon cancer |
| VEGFR2 (KDR) | 11/12 or 11/11 AC repeats | Tumor recurrence in stage |
| at position 4422 AC repeat | | II colon cancer |
| EGFR Intron I at position | 2 alleles with <20 CA repeats | Tumor recurrence in stage |
| 496 CA repeats | | II colon cancer |
| AM 3′UTR CA repeat | 2 alleles with <14 CA repeats | Tumor recurrence in stage |
| | 2 alleles with ≧14 CA repeats | II colon cancer |
| IL-1β (3954 C/T) | C/C or C/T | Tumor recurrence in stage |
| | | II colon cancer |
| VEGF (936 C/T) and | T/T in VEGF and | Tumor recurrence in stage |
| IL-8 (−251 T/A) | T/T in IL-8 | III colon cancer |
| VEGF (936 C/T) and | C/C in VEGF and | Tumor recurrence in stage |
| IL-8 (−251 T/A) | T/T or T/A in IL-8 | III colon cancer |
| VEGF (936 CTT) and | C/T or T/T in VEGF and | Tumor recurrence in stage |
| IL-8 (−251 T/A) | A/A in IL-8 | III colon cancer |

TABLE 2
Additional Polymorphisms Assayed

| | Predictive Genotype | |
|---|---|---|
| Allele (Polymorphism) | (Polymorphism) | Measured Response |
| EGF | 61 A/G | No Correlation |
| ARNT | Exon 8 G/C | No Correlation |
| Hif-1α | 1772 C/T | No Correlation |
| TGF-β | 29 T/C | No Correlation |
| NRP-1 | 3′ end C/T | No Correlation |
| Leptin | −2548 G/A | No Correlation |
| VEGF | 634 G/C | No Correlation |
| PLGF | 3′ UTR G/A or 3′ UTR T/A | No Correlation |
| CXCR1 | 2607 G/C | No Correlation |
| CXCR2 | 785 C/T | No Correlation |
| IGF2 | 4205 G/A | No Correlation |
| IL-6 | 174 G/C | No Correlation |
| FGFR4 | 388 G/A | No Correlation |
| IGFBP | 2133 G/C | No Correlation |
| COX-2 | 3′UTR 8473 T/C | No Correlation |
| ICAM | 241 G/A | No Correlation |
| E-cadherin | −160 C/A | No Correlation |
| TF | −603 A/G | No Correlation |
| MDM-2 | 309 T/G | No Correlation |
| GLUT-1 | 5′ UTR-2841 A/T | No Correlation |
| LDH-5 | Exon 5 C/T or G/A | No Correlation |
| SDF1 | 3′ UTR G/A | No Correlation |
| MMP-2 | −1306 C/T | No Correlation |
| MMP-7 | −181 A/G | No Correlation |
| MMP-9 | −1562 C/T | No Correlation |
| Survivin | 31 G/C | No Correlation |
| ADAM10 | 5′UTR G/A | No Correlation |
| ADAM17 | 3′UTR G/A | No Correlation |

For patients screened using methods known in the art and described herein and having the genetic polymorphism as identified in the center column of Table 1, this invention also provides methods for treating these patients by administering an effective amount of a pyrimidine based antimetabolite and an efficacy enhancing agent based chemotherapy. In another aspect, the chemotherapy comprises, or alternatively consists essentially of, or yet further consists of, the administration of an effective amount of radiation therapy. In another aspect it comprises, or alternatively consists essentially of, or yet further consists of, administration of an effective amount of a platinum-based alkylating agent. In yet another aspect, the chemotherapy comprises, or alternatively consists essentially of, or yet further consists of, the administration of an effective amount of a topoisomerase I inhibitor. These therapies can be administered alone or in combination with each other. Such combinations are known to the skilled artisan and are sometimes referred to as 5-FU adjuvant therapy.

The various embodiments are set forth herein.

In one aspect, the invention is a method for identifying responsiveness to a pyrimidine based antimetabolite and an efficacy enhancing agent based chemotherapy by assaying a suitable patient sample from a patient suffering from a solid malignant tumor, gastrointestinal cancer or lung cancer, for at least one polymorphism identified in Table 1, above. Patients having at least one genotype selected from (C/T or T/T) for VEGF at nt 936 C/T (also defined herein as VEGF +936 or VEGF C+936T); (T/T or T/A) for IL-8 at nt −251 T/A; (11/12 or 11/11 AC repeats) for VEGFR2 (KDR) at position 4422; (2 alleles with <20 CA repeats) for EGFR at 496 (CA repeats in Intron I); (2 alleles with <14 CA repeats or 2 alleles with ≧14 CA repeats) for AM 3′UTR CA repeat; or (C/C or C/T) for IL-1β at nt 3954 C/T; each of (T/T) for VEGF at nt 936 C/T and (T/T) for IL-8 at nt −251 T/A; each of (C/C) for VEGF at nt 936 C/T and (T/T or T/A) for IL-8 at nt −251 T/A; or each of (C/T or T/T) for VEGF at nt 936 C/T and (A/A) for IL-8 at nt −251 T/A, are likely to show responsiveness to combination pyrimidine based antimetabolites and efficacy enhancing agent base chemotherapy, wherein responsiveness is selected from the group of clinical parameters of reduction in tumor load or size, time to tumor-free progression, lack of tumor recurrence in stage II or III colon or colorectal cancer or overall survival. In one aspect, the patient has previously undergone surgical resection to remove the tumor mass or alternatively has undergone a lymphectomy.

In another aspect, the chemotherapy may comprise the administration of an effective amount of a platinum-based alkylating agent.

In another aspect, the chemotherapy may comprise the administration of an effective amount of a topoisomerase I inhibitor.

In another aspect, the patient is suffering from a solid malignant tumor such as a gastrointestinal cancer or lung cancer or tumor, e.g., rectal cancer, colorectal cancer, colon cancer, gastric cancer, lung cancer, non-small cell lung cancer and esophageal cancer. In a particular embodiment, the patient is suffering from colorectal cancer or colon cancer. In an alternative aspect, the patient is suffering one of the above cancers that is metastatic, e.g., from metastatic colorectal or metastatic colon cancer. In a further aspect, the patient is suffering from stage II or stage III disease, e.g., stage III colon cancer.

In another aspect, the patient is diagnosed with stage II colon cancer and wherein the presence of at least one of the following said respective genetic polymorphism identifies the patient as likely to respond to said therapy: (11/12 or 11/11 AC repeats) for VEGFR2 (KDR) at position 4422; (2 alleles having <20 CA repeats) for EGFR at 496 CA repeats in Intron I; (2 alleles with <14 CA repeats or 2 alleles with ≧14 CA repeats) for AM 3′UTR CA repeat; or (C/C or C/T) IL-10 at nt 3954 C/T. In one aspect, at least one genetic polymorphism of (11/12 AC repeats) for VEGFR2 (KDR) at position 4422; or 2 alleles with <14 CA repeats for AM 3′UTR CA repeat; or (C/C) at IL-1β at nt 3954 C/T, identifies the patient as most likely to respond to said therapy.

In yet a further aspect, the patient is diagnosed with stage III colon cancer and wherein the presence of at least one of the following said respective genetic polymorphism identifies the patient as likely to respond to said therapy: (C/T or T/T) for VEGF at nt 936 C/T; (T/T or T/A) for IL-8 at nt −251 T/A; (T/T) for VEGF at nt 936 C/T and (T/T) for IL-8 at nt −251 T/A; (C/C) for VEGF at nt 936 C/T and (T/T or T/A) for IL-8 at nt −251 T/A; or (C/T or T/T) for VEGF at nt 936 C/T and (A/A) for IL-8 −251 T/A. In one aspect, at least one genetic polymorphism of (T/T) for IL-8 at nt −251 T/A or (T/T) for VEGF at nt 936 C/T and (T/T) for IL-8 −251 T/A, identifies the patient as most likely to respond to said therapy.

To practice the above methods, the sample is a patient sample containing the tumor tissue, normal tissue adjacent to said tumor, normal tissue distal to said tumor or peripheral blood lymphocytes. In one aspect, the method also requires isolating a sample containing the genetic material to be tested; however, it is conceivable that one of skill in the art will be able to analyze and identify genetic polymorphisms in situ at some point in the future. Accordingly, the inventions of this application are not to be limited to requiring isolation of the genetic material prior to analysis.

These methods are not limited by the technique that is used to identify the polymorphism of interest. Suitable methods include, but are not limited to, the use of hybridization probes, antibodies, primers for PCR analysis and gene chips, slides and software for high throughput analysis. Additional polymorphisms can be assayed and used as negative controls which include, but are not limited to those identified in Table 2, above.

After a patient has been identified as positive for one or more of the polymorphisms identified in Table 1, or a method identified above, the method may comprise, or alternatively consists essentially of, or yet further consists of, administering or delivering an effective amount of a pyrimidine based antimetabolite and an efficacy enhancing agent based chemotherapy for treatment. In a further aspect, the method may comprise, or alternatively consist essentially of, or yet further consist of, administering or delivering an effective amount of platinum-based alkylating agent. In yet another aspect, the method may comprise, or alternatively consist essentially of, or yet further consist of, administering or delivering an effective amount of a topoisomerase I inhibitor. Methods of administering these pharmaceuticals are known in the art and incorporated herein by reference.

In another aspect, alternative genetic polymorphisms identified in Table 2 can be used as negative controls for a patient who will not likely show responsiveness to the therapies described herein. Patients having genetic polymorphisms selected from at least one, or alternatively at least two, or alternatively at least three, or alternatively at least four, or alternatively at least five, or alternatively at least six, or alternatively at least seven, or alternatively at least eight, or alternatively at least nine, or alternatively at least ten, or alternatively at least eleven, or alternatively at least twelve, or alternatively at least thirteen, or alternatively at least fourteen, or alternatively at least fifteen, or alternatively at least sixteen, or alternatively at least seventeen, or alternatively at least eighteen, or alternatively at least nineteen, or alternatively at least twenty, or alternatively at least twenty-one, or alternatively at least twenty-two, or alternatively at least twenty-three, or alternatively at least twenty-four, or alternatively at least twenty-five, or alternatively at least twenty-six, or alternatively at least twenty-seven, or alternatively all twenty-eight of (61 A/G) of EGF; (G/C in Exon 8) of ARNT; (1772 C/T) of Hif-1α; (29 T/C) of TGF-β; (3′ end C/T) of NRP-1; (−2548 G/A) of Leptin; (634 G/C) of VEGF; (3′UTR G/A or 3′UTR T/A) of PLGF; (2607 G/C) of CXCR1; (785 C/T) of CXCR2; (4205 G/A) of IGF2; (174 G/C) of IL-6; (388 G/A) of FGFR4; (2133 G/C) of IGFBP; (3′UTR 8473 T/C) of COX-2; (241 G/A) of ICAM; (−160 C/A) of E-cadherin; (−603 A/G) of TF; (309 T/G) of MDM-2; (5′UTR -2841 A/T) of GLUT-1; (Exon 5 C/T or G/A) of LDH-5; (3′UTR G/A) of SDF1; (−1306 C/T) of MMP-2; (−181 A/G) of MMP-7; (−1562 C/T) of MMP-9; (31 G/C) of Survivin; (5′UTR G/A) of ADAM10; or (3′UTR G/A) of ADAM17, will unlikely show responsiveness, wherein responsiveness is selected from the group of clinical parameters of reduction in tumor load or size, time to tumor-free progression, lack of tumor recurrence in stage II or III colorectal cancer or overall survival. In one aspect, the patient has previously undergone surgical resection to remove the tumor mass, or a lymphectomy.

The invention also is a method for identifying a patient likely or more likely responsive to a therapy and/or selecting a chemotherapy comprising, or alternatively consisting essentially of, or consisting of, a pyrimidine based antimetabolite and an efficacy enhancing agent based chemotherapy by assaying a suitable patient sample from a patient suffering from a solid malignant tumor or gastrointestinal cancer, for at least one polymorphism identified in Table 1, above. This invention also provides a method for selecting a chemotherapy for a gastrointestinal or lung cancer patient in need of additional therapy and is most likely to benefit from pyrimidine based antimetabolite and efficacy enhancing agent based chemotherapy, comprising screening a suitable cell or tissue sample isolated from said patient for at least one genetic polymorphism selected from that identified in Table 1, above.

Patients who are considered positive responders for this or further pyrimidine based antimetabolite and an efficacy enhancing agent based therapy have at least one genotype selected from (C/T or T/T) for VEGF at nt 936 C/T; (T/T or T/A) for IL-8 at nt −251 T/A; (11/12 or 11/11 AC repeats) for VEGFR2 (KDR) at position 4422; (2 alleles with <20 CA repeats) for EGFR at 496 CA repeats in Intron I; (2 alleles with <14 CA repeats or 2 alleles with ≧14 CA repeats) for AM 3′UTR CA repeat; (C/C) IL-1β at nt 3954 C/T; each of (T/T) for VEGF at nt 936 C/T and (T/T) for IL-8 at nt −251 T/A; each of (C/C) for VEGF at nt 936 C/T and (T/T or T/A) for IL-8 at nt −251 T/A; or each of (C/T or T/T) for VEGF at nt 936 C/T and (A/A) for IL-8 at nt −251 T/A. In one aspect, at least one genetic polymorphism of (11/12 AC repeats) for VEGFR2 (KDR) at position 4422; or 2 alleles with <14 CA repeats for AM 3′UTR CA repeat; or (C/C) at IL-1β at nt 3954 C/T, identifies the patient as most likely to respond to said therapy. In one aspect, at least one genetic polymorphism of (T/T) for IL-8 at nt −251 T/A or (T/T) for VEGF at nt 936 C/T and (T/T) for IL-8 −251 T/A, identifies the patient as most likely to respond to said therapy. These patients show responsiveness to pyrimidine based antimetabolite and an efficacy enhancing agent based therapy, wherein responsiveness is selected from the group of clinical parameters of reduction in tumor load or size, time to tumor-free progression, lack of tumor recurrence or a delay in time to tumor recurrence, or enhanced overall survival. In one aspect, the patient is suffering from a solid malignant tumor such as a gastrointestinal or lung tumor, e.g., from rectal cancer, colorectal cancer, metastatic colorectal cancer, colon cancer, gastric cancer, lung cancer, non-small cell lung cancer and esophageal cancer. In a further aspect, the patient has been diagnosed with stage II or stage III cancer, e.g., colorectal and/or colon cancer.

In another aspect, the chemotherapy may comprise the administration of an effective amount of a platinum-based alkylating agent.

In another aspect, the chemotherapy may comprise, or alternatively consist essentially of, or alternatively consist of, administration of an effective amount of a topoisomerase I inhibitor.

To practice these methods, the sample is a patient sample containing the tumor tissue, normal tissue adjacent to said tumor, normal tissue distal to said tumor or peripheral blood lymphocytes. These methods are not limited by the technique that is used to identify the polymorphism of interest. Suitable methods include but are not limited to the use of hybridization probes, antibodies, primers for PCR analysis and gene chips and software for high throughput analysis. Additional polymorphisms can be assayed and used as negative controls. These additional polymorphisms can include, but are not limited to those identified in Table 2, above.

In one aspect, the method also requires isolating a sample containing the genetic material to be tested; however, it is conceivable that one of skill in the art will be able to analyze and identify genetic polymorphisms in situ at some point in the future. Accordingly, the inventions of this application are not to be limited to requiring isolation of the genetic material prior to analysis.

After a patient has been identified as positive for one or more of the polymorphisms identified in Table 1, the method may further comprise, or alternatively further consist essentially of, or yet further consist of, administering or delivering an effective amount of a pyrimidine based antimetabolite and an efficacy enhancing agent based chemotherapy to the patient. In a further aspect, the method may comprise, or consist essentially of, or further consist of, the administering or delivering an effective amount of platinum-base alkylating agent. In yet another aspect, the method may comprise, or alternatively consist of or yet further, consist of, the administering or delivering an effective amount of a topoisomerase I inhibitor. Methods of administration of these pharmaceuticals are known in the art and incorporated herein by reference.

The invention is a method for treating a gastrointestinal or lung cancer patient identified as having at least one genetic polymorphism identified in the center column of Table 1, above. Patients who are considered positive responders for further pyrimidine based antimetabolites and efficacy enhancing agent therapy have at least one, or alternatively at least two, or alternatively at least three, or alternatively at least four, or alternatively at least five, or alternatively at least six, or alternativetively all seven, or alternatively all eight, or alternatively all nine genetic polymorphisms selected from (C/T or T/T) for VEGF at nt 936 C/T; (T/T or T/A) for IL-8 at nt −251 T/A; (11/12 or 11/11 AC repeats) for VEGFR2 (KDR) at position 4422; (2 alleles with <20 CA repeats) for EGFR at 496 CA repeats in Intron I; (2 alleles with <14 CA repeats or 2 alleles with ≧14 CA repeats) for AM 3′UTR CA repeat; (C/C or C/T) IL-1β at nt 3954 C/T; each of (C/C) for VEGF at nt 936 C/T and (T/T or T/A) for IL-8 at nt −251 T/A; each of (T/T) for VEGF at nt 936 C/T and (T/T) for IL-8 at nt −251 T/A; or each of (C/T or T/T) for VEGF at nt 936 C/T and (A/A) for IL-8 at nt −251 T/A. In one particular aspect, the patient has each of (C/T or T/T) for VEGF at nt 936 C/T and (T/T or T/A) for IL-8 at nt −251 T/A. In one aspect, at least one genetic polymorphism of (11/12 AC repeats) for VEGFR2 (KDR) at position 4422; or 2 alleles with <14 CA repeats for AM 3′UTR CA repeat; or (C/C) at IL-1βat nt 3954 C/T, identifies the patient as most likely to respond to said therapy. In one aspect, at least one genetic polymorphism of (T/T) for IL-8 at nt −251 T/A or (T/T) for VEGF at nt 936 C/T and (T/T) for IL-8 −251 T/A, identifies the patient as most likely to respond to said therapy. These patients show responsiveness to administration of a pyrimidine based antimetabolite and an efficacy enhancing agent, wherein responsiveness is selected from the group of clinical parameters of reduction in tumor load or size, time to tumor-free progression, a delay or lack of tumor recurrence or enhanced overall survival. In one aspect responsiveness is measured in a delay in time to tumor recurrence for patients suffering from stage II or III colorectal cancer. In one aspect, the patient has previously undergone surgical resection to remove the tumor mass or a lymphectomy.

After a patient has been identified as positive for one or more of the polymorphisms identified in Table 1, the method may further comprise, or alternatively consist essentially of, or yet further consist of, administering or delivering an effective amount of a pyrimidine based antimetabolite and an efficacy enhancing agent based chemotherapy for treatment. In a further aspect, the method may comprise, or alternatively consist essentially of, or yet further consist of, the administering or delivering an effective amount of platinum-base alkylating agent. In yet another aspect, the method may comprise, or alternatively consist essentially of, or yet further consist of, the administering or delivering an effective amount of a topoisomerase I inhibitor. Methods of administration of these pharmaceuticals are known in the art and incorporated herein by reference.

In another aspect, the patient is suffering from a solid malignant tumor such as a gastrointestinal or lung tumor, e.g., from rectal cancer, colorectal cancer, metastatic colorectal cancer, colon cancer, gastric cancer, lung cancer, non-small cell lung cancer and esophageal cancer. In a further aspect, the tumor or neoplasm is colon cancer or colorectal cancer.

To practice this method, the sample is a patient sample containing the tumor tissue, normal tissue adjacent to said tumor, normal tissue distal to said tumor or peripheral blood lymphocytes.

In one aspect, the method also requires isolating a sample containing the genetic material to be tested; however, it is conceivable that one of skill in the art will be able to analyze and identify genetic markers in situ at some point in the future. Accordingly, the inventions of this application are not to be limited to requiring isolation of the genetic material prior to analysis.

These methods also are not limited by the technique that is used to identify the polymorphism of interest. Suitable methods include but are not limited to the use of hybridization probes, antibodies, primers for PCR analysis, and gene chips, slides and software for high throughput analysis. Additional genetic polymorphisms can be assayed and used as negative controls.

In an alternate aspect of the method described above, the method identifies the patients more likely to show responsiveness to the chemotherapy described herein. The patients more likely to show responsiveness have one or more of each of (T/T) in VEGF 936 and (T/T) in IL-8; or (T/T) for IL-8 at nt −251 T/A; (11/12 AC repeats) for VEGFR2 (KDR) at position 4422; or (2 alleles w/<14 CA repeats) for AM 3′UTR CA repeat, or (C/C) IL-1β at nt 3954 C/T. Having being identified as more likely to benefit from the therapy because of the delay in time to tumor recurrence, these patients can be selected for the therapy as described herein. For stage III cancer patients, VEGF 936 (C/T) and IL-8 −251 (T/A) were better prognostic markers for time to tumor recurrence. VEGFR2 AC repeat; or EGFR CA repeats; or AM AC repeat or IL-1B 3954 C/T, each as described in the center column of Table 1, were better prognostic markers for time to tumor recurrence for stage II cancer patients. In one aspect, at least one genetic polymorphism of (11/12 AC repeats) for VEGFR2 (KDR) at position 4422; or 2 alleles with <14 CA repeats for AM 3′UTR CA repeat; or (C/C) at IL-1β at nt 3954 C/T, identifies the patient as most likely to respond to said therapy. In one aspect, at least one genetic polymorphism of (T/T) for IL-8 at nt −251 T/A or (T/T) for VEGF at nt 936 C/T and (T/T) for IL-8 −251 T/A, identifies the patient as most likely to respond to said therapy. Alleles coding for less expression within the VEGFR2 and EGFR microsatellite polymorphisms also as identified on Table 1, were associated with less favorable outcome.

Alternatively, the invention is a method of identifying a lung cancer or gastrointestinal cancer patient who is less likely to benefit from pyrimidine based antimetabolite and an efficacy enhancing agent based chemotherapy or alternatively, in need of additional therapy. Patients who are considered more likely to have tumor recurrence are therefore in need of additional or alternate therapy, and therefore are selected by having the genetic polymorphisms of at least one, or alternatively at least two, or alternatively at least three, or alternatively at least four, or alternatively at least five, or alternatively at least six, or alternatively all seven polymorphisms selected from (C/C) for VEGF at nt 936 C/T; (A/A) for IL-8 at nt −251 T/A; (12/12 AC repeats) for VEGFR2 (KDR) at position 4422; (at least 1 allele with ≧20 CA repeats) for EGFR at 496 CA repeats in Intron I; (only 1 allele with ≧14 CA repeats) for AM 3′UTR CA repeat; (T/T) IL-1β at nt 3954 T/T; or (C/C) for VEGF at nt 936 C/T and (A/A) for IL-8 at nt −251 T/A. These patients are at higher risk for less time to tumor recurrence after therapy and therefore are likely in need of additional or alternate therapy. In one aspect, the patient may receive a pyrimidine based antimetabolite and an efficacy enhancing agent based chemotherapy or other therapy.

In another aspect, the patient is diagnosed with stage II colon cancer and wherein the presence of at least one of the following said respective genetic polymorphism identifies the patient as in need of additional chemotherapy or less likely to respond to said therapy: (12/12 AC repeats) for VEGFR2 (KDR) at position 4422; (at least one allele with ≧20 CA repeats) for EGFR at 496 CA repeats in Intron I; (only 1 allele with ≧14 CA repeats) for AM 3′UTR CA repeat; or (T/T) IL-1β at nt 3954 C/T.

In another aspect, the patient is diagnosed with stage III colon cancer and wherein the presence of at least one of the following said respective genetic polymorphism identifies the patient as in need of additional chemotherapy or less likely to respond to said therapy: (C/C) for VEGF at nt 936 C/T; (A/A) for IL-8 at nt −251 T/A; or (C/C) for VEGF at nt 936 C/T and (A/A) for IL-8 at nt −251 T/A.

In another aspect, the chemotherapy may comprise the administration of an effective amount of a platinum-based alkylating agent.

In another aspect, the chemotherapy may comprise the administration of an effective amount of a topoisomerase I inhibitor.

In one aspect, the patient is suffering from a solid malignant tumor such as a lung or gastrointestinal tumor, e.g., from rectal cancer, colorectal cancer, metastatic colorectal cancer, colon cancer, gastric cancer, lung cancer, non-small cell lung cancer and esophageal cancer.

To practice this method, the sample is a patient sample containing the tumor tissue, normal tissue adjacent to said tumor, normal tissue distal to said tumor or peripheral blood lymphocytes. These methods are not limited by the technique that is used to identify the polymorphism of interest. Suitable methods include but are not limited to the use of hybridization probes, antibodies, primers for PCR analysis and gene chips and software for high throughput analysis. Additional polymorphisms can be assayed and used as negative controls. These additional polymorphisms can include, but are not limited to those identified in Table 2, above.

In one aspect, the method also requires isolating a sample containing the genetic material to be tested; however, it is conceivable that one of skill in the art will be able to analyze and identify genetic polymorphisms in situ at some point in the future. Accordingly, the inventions of this application are not to be limited to requiring isolation of the genetic material prior to analysis.

After a patient has been identified as positive for one or more of the alternative genotypes identified above, the method may further comprise, or alternatively consist essentially of, or yet further consist of, administering or delivering an effective amount of a pyrimidine based antimetabolite and an efficacy enhancing agent based chemotherapy for treatment. In a further aspect, the method may comprise, or alternatively consist essentially of, or yet further consist of, the administering or delivering an effective amount of platinum-base alkylating agent. In yet another aspect, the method may comprise, or alternatively consist essentially of, or yet further consist of, the administering or delivering an effective amount of a topoisomerase I inhibitor. Methods of administration of these pharmaceuticals are known in the art and incorporated herein by reference.

In a further aspect, the invention is a method comprising, or alternatively consisting essentially of, or yet further consisting of, comparing the genotype of a patient against the identified genotypes of Tables 1 and 2 alone, or a combination of Tables 1 and 2. Suitable patients for the method are those having a lung or gastrointestinal malignant tumor. If a patient has a genotype matching at least one, or alternatively at least two, or alternatively at least three, or alternatively at least four, or alternatively at least five, or or alternatively at least six, or alternatively all seven genetic polymorphisms of Table 1 alone or in combination with at least one, or alternatively at least two, or alternatively at least three, or alternatively at least four, or alternatively at least five, or alternatively at least six, or alternatively at least seven, or alternatively at least eight, or alternatively at least nine, or alternatively at least ten, or alternatively at least eleven, or alternatively at least twelve, or alternatively at least thirteen, or alternatively at least fourteen, or alternatively at least fifteen, or alternatively at least sixteen, or alternatively at least seventeen, or alternatively at least eighteen, or alternatively at least nineteen, or alternatively at least twenty, or alternatively at least twenty-one, or alternatively at least twenty-two, or alternatively at least twenty-three, or alternatively at least twenty-four, or alternatively at least twenty-five, or alternatively at least twenty-six, or alternatively at least twenty-seven, or alternatively all twenty-eight of Table 2, then an effective amount of a pyrimidine based antimetabolite and an efficacy enhancing agent is administered or delivered to the patient. In some aspects of the invention, effective amount of a platinum-based alkylating agent is administered or delivered to the patient. In yet other aspects of the invention, effective amounts of a topoisomerase I inhibitor is administered or delivered to the patient. This invention also provides the step of administration or delivery of said therapy.

In each of the above aspects, examples of pyrimidine based antimetabolites are selected from the group, but are not limited to Fluorouracil (5-FU) and Capecitabine (XEL) or chemical equivalents thereof In a further aspect of the invention, the efficacy enhancing agent of the pyrimidine based antimetabolites is Leucovorin or a chemical equivalent thereof.

In each of the above aspects, an example of a platinum-based alkylating agent is, but not limited to Oxaliplatin (OX) or a chemical equivalent thereof

In each of the above aspects, an examples of topoisomerase I inhibitors are selected from the group, but not limited to Irinotecan, CPT-11, Camptosar or a chemical equivalent thereof.

In each of the above aspects, the administration of a pyrimidine based antimetabolite, an efficacy enhancing agent, and a platinum-based alkylating agent are selected from the group, but not limited to Fluorouracil, Leucovorin, and Oxaliplatin (FOLFOX) or chemical equivalents thereof.

This invention also provides a panel, kit, software and/or gene chip for patient sampling and performance of the methods of this invention. The kits contain panels, gene chips, probes and/or primers that can be used to amplify and/or for determining the molecular structure of the polymorphisms identified above. In an alternate embodiment, the kit contains antibodies and/or other polypeptide binding agents that are useful to identify a polymorphism of Tables 1 and/or 2 alone or in combination. Instructions for using the materials to carry out the methods are further provided.

The present invention provides methods and kits for identifying patients having solid malignant tumor masses or cancers who are likely to respond to combination pyrimidine-based antimetabolite and platinum-based alkylating agent based chemotherapy. The methods require determining the subject's genotype at the gene of interest. Other aspects of the invention are described below or will be apparent to one of skill in the art in light of the present disclosure.

This invention also provides for a prognostic panel of genetic markers selected from, but not limited to the genetic polymorphisms identified in Tables 1 and 2 alone or in combination. The prognostic panel comprises probes or primers that can be used to amplify and/or for determining the molecular structure of the polymorphisms identified above. The probes or primers can be attached or supported by a solid phase support such as, but not limited to a gene chip or microarray. The probes or primers can be detectably labeled. This aspect of the invention is a means to identify the genotype of a patient sample for the genes of interest identified above.

### BRIEF DESCRIPTION OF THE FIGURES

Seven figures are attached to this application. The figures graphically illustrates the results of the experimental example.

FIG. 1 shows the VEGF allele polymorphism 936 C/T predicts tumor recurrence in Stage III colorectal cancer and more particularly Stage III colon cancer.

FIG. 2 shows the IL-8 allele polymorphism −251 T/A predicts tumor recurrence in Stage III colorectal cancer and more particularly Stage III colon cancer.

FIG. 3 shows the VEGFR2 (KDR) allele polymorphism 4422 AC repeat predicts tumor recurrence in Stage II colorectal cancer and more particularly Stage II colon cancer.

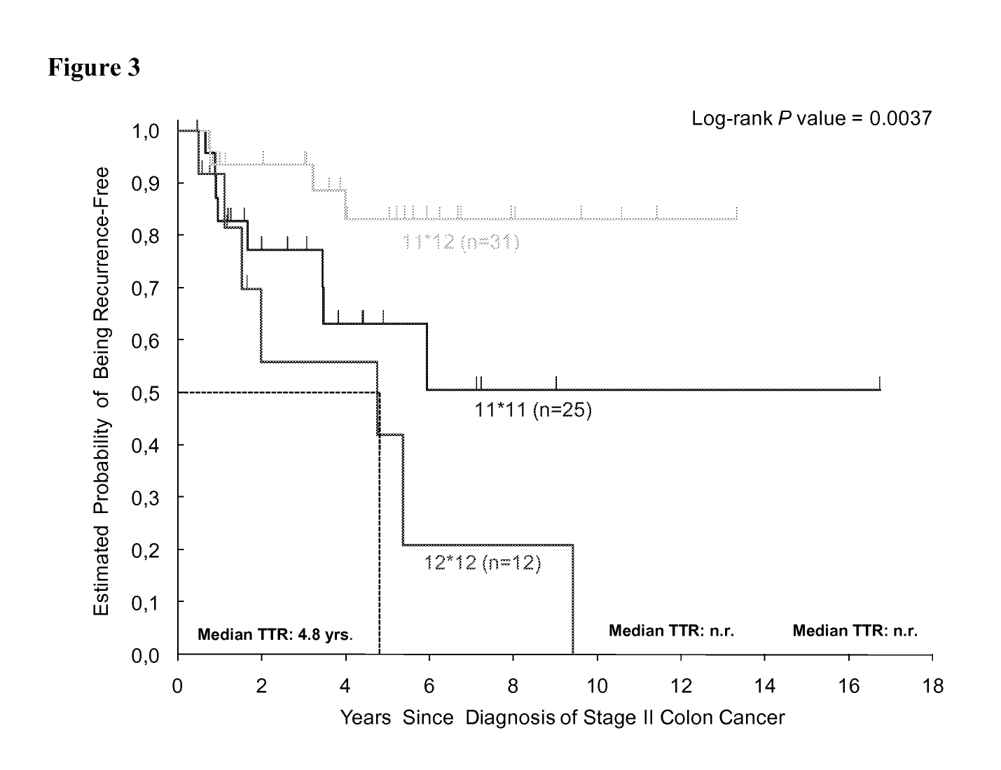

FIG. 4 shows the EGFR allele polymorphism at position 496 CA repeats of Intron I predicts tumor recurrence in Stage II colorectal cancer and more particularly Stage II colon cancer.

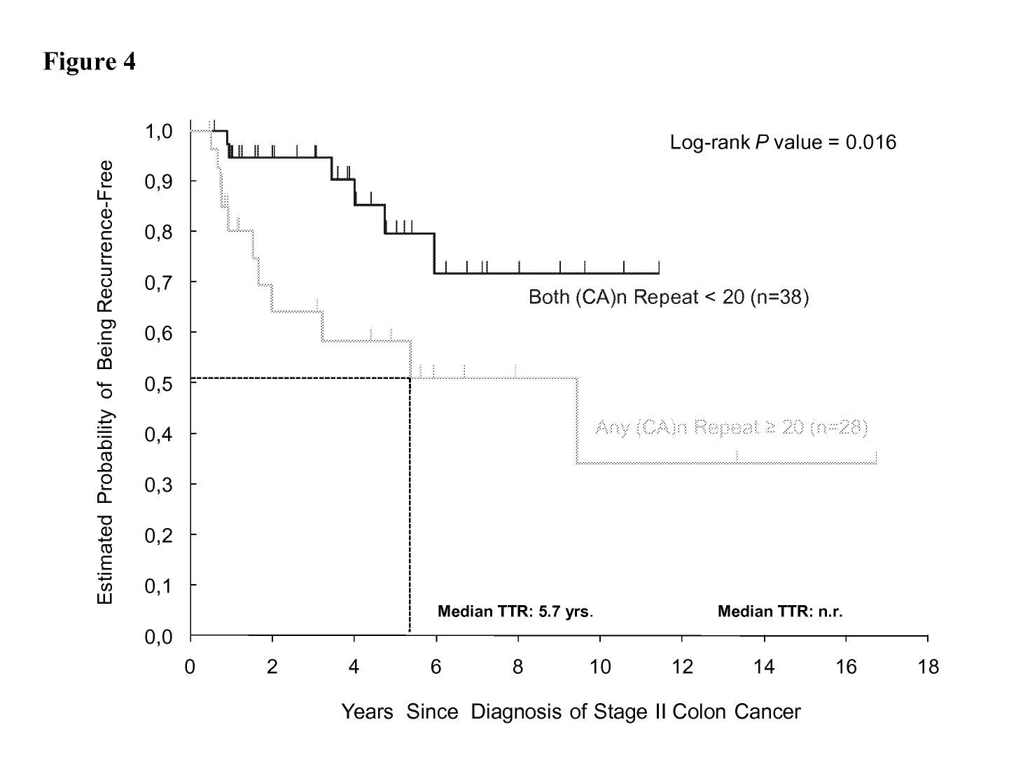

FIG. 5 shows the AM allele 3′UTR CA repeat polymorphism predicts tumor recurrence in Stage II colorectal cancer and more particularly Stage II colon cancer.

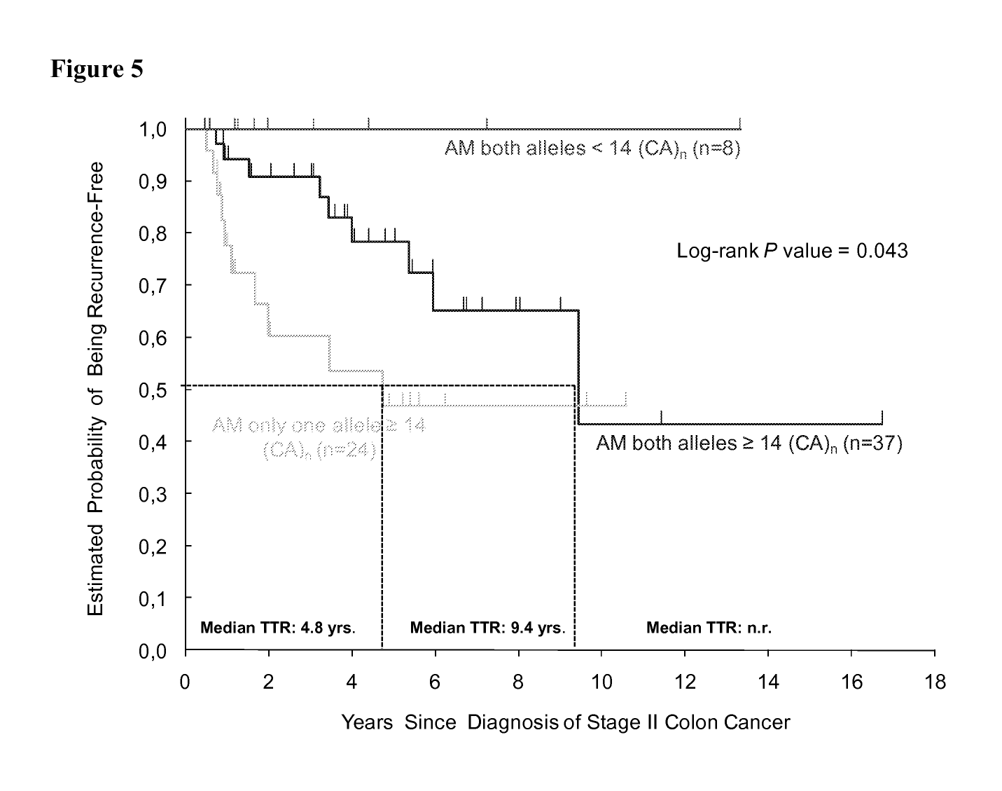

FIG. 6 shows the IL-1β allele polymorphism 3954 C/T predicts tumor recurrence in Stage II colorectal cancer and more particularly Stage II colon cancer.

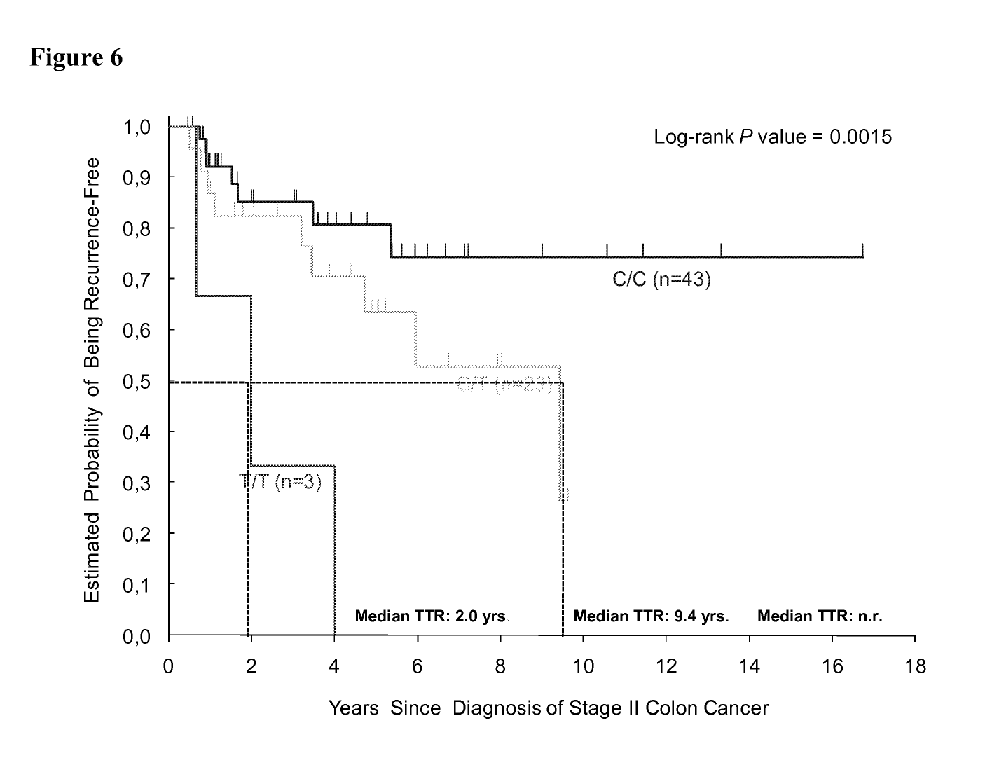

FIG. 7 shows that recurrence-free survival of patients with Stage III colon cancer by combination of VEGF and IL-8 polymorphisms. Verical hash marks show time of last follow-up for those patients who were still recurrence-free at the time of the analysis of data. All censored patients and those who were recurrent are accounted for.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)); MODERN EPIDEMIOLOGY (Rothman, ed., Lippincott-Raven (1998)); MANIPULATING THE MOUSE EMBRYO: A LABORATORY MANUAL 3^{rd} edition (Cold Spring Harbor Laboratory Press, Cold Spring Harbor, N.Y. (2002)).

### DEFINITIONS

As used herein, certain terms may have the following defined meanings As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of when used to define compositions and methods, shall mean excluding other elements of any essential significance to the composition or method. “Consisting of shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively include additional steps and compositions of no significance (consisting essentially of) or alternatively, intending only the stated methods steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X +0.1” or “X −0.1.” It also is to be understood, although not always explicitly stated, that the reagents described herein are merely exemplary and that equivalents of such are known in the art.

Fluorouracil (5-FU) belongs to the family of chemotherapy drugs called pyrimidine based antimetabolites. It is a pyrimidine based analog, which is transformed into different cytotoxic metabolites that are then incorporated into DNA and RNA thereby inducing cell cycle arrest and apoptosis. Similar to 5-FU, chemical equivalents are pyrimidine analogs which result in disruption of DNA replication. Chemical equivalents inhibit cell cycle progression at S phase resulting in the disruption of cell cycle and consequently apoptosis. Chemical equivalents are know in the art and are described, for example in Papamichael (2000) Stem Cells 18:166-175.

Capecitabine is an example of a chemical equivalent of 5-FU. It is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is an adjuvant used in cancer chemotherapy. It is used in synergistic combination with 5-FU to improve efficacy of the chemotherapeutic agent. Efficacy of 5-FU is enhanced with addition of Leucovorin by inhibiting thymidylate synthase.

Oxaliplatin belongs to a family of platinum-based chemotherapy drugs. Its mechanism of action is currently unknown. Its anti-tumor activity against colon carcinoma is through non-targeted cytotoxic effects. Chemical equivalents are compounds based on platinum derived alkylating agents which result in cancer cell toxicity and death.

Irinotecan (CPT-11) is sold under the tradename of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

In one aspect, the “chemical equivalent” means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

The terms “protein,” “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “allelic variant of a polymorphic region of the gene of interest” refers to a region of the gene of interest having one of a plurality of nucleotide sequences found in that region of the gene in other individuals.

As used herein, the term “gene of interest” intends one or more genes selected from the group consisting of VEGF, IL-8, VEGFR2 (KDR), EGFR, IL-1β, EGF, ARNT, Hif-1α, TGF-β, NRP-1, Leptin, AM, PLGF, CXCR1, CXCR2, IGF2, IL-6, FGFR4, IGFBP, COX-2, ICAM, E-cadherin, TF, MDM-2, GLUT-1, LDH-5, SDF1, MMP-2, MMP-7, MMP-9, Survivin, ADAM10, ADAM17.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “genotype” refers to the specific allelic composition of an entire cell or a certain gene, whereas the term “phenotype' refers to the detectable outward manifestations of a specific genotype.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “mismatches” refers to hybridized nucleic acid duplexes which are not 100% homologous. The lack of total homology may be due to deletions, insertions, inversions, substitutions or frameshift mutations.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be

DNA or an RNA, the terms “adenosine,” “cytidine,” “guanosine,” and “thymidine” are used. It is understood that if the nucleic acid is RNA, a nucleotide having a uracil base is uridine.

The terms “oligonucleotide” or “polynucleotide,” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

A “polymorphic gene” refers to a gene having at least one polymorphic region.

When a genetic marker or polymorphism “is used as a basis” for selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease. For example, in the case of cancer, treatment includes a reduction in cachexia, increase in survival time, elongation in time to tumor progression, reduction in tumor mass, reduction in tumor burden and/or a prolongation in time to tumor metastasis, each as measured by standards set by the National Cancer Institute and the U.S. Food and Drug Administration for the approval of new drugs. See Johnson et al. (2003) J. Clin. Oncol. 21(7) :1404-1411.

The term “likely to respond” shall mean that the patient is more likely than not to exhibit at least one of the described clinical parameters or treatment responses, identified above, as compared to similarly situated patients without the polymorphism. Alternatively, “less likely to respond” indicates the patient is less likely than not to exhibit at least one of the described clinical parameters or treatment responses, identified above, as compared to similarly situated patients without the polymorphism.

“An effective amount” intends to indicated the amount of a compound or agent administered or delivered to the patient which is most likely to result in the desired response to treatment. The amount is empirically determined by the patient's clinical parameters including, but not limited to the stage of disease, age, gender, histology, and likelihood for tumor recurrence.

“Progression free survival” (PFS) indicates the length of time during and after treatment that the cancer does not grow. Progression-free survival includes the amount of time patients have experienced a complete response or a partial response, as well as the amount of time patients have experienced stable disease.

A “complete response” (CR) to a therapy defines patients with evaluable but non-measurable disease, whose tumor and all evidence of disease had disappeared.

A “partial response” (PR) to a therapy defines patients with anything less than complete response were simply categorized as demonstrating partial response.

“Stable disease” (SD) indicates that the patient is stable.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“No Correlation” refers to a statistical analysis showing no relationship between the allelic variant of a polymorphic region and clinical parameters.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

As used herein, the terms “stage II cancer” and “stage III cancer” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

The term “clinical parameters” refers to a reduction or delay in recurrence of the cancer after the initial therapy, time to tumor recurrence (TTR), time to tumor progression (TTP), decrease in tumor load or size (tumor response or TR), progression free survival, increase median survival time (OS) or decrease metastases.

In one aspect, the therapy to be selected or administered to a patient is one that comprises, or alternatively consists essentially of, or yet further consists of a combination of pyrimidine based antimetabolite and an efficacy enhancing agent. One example of such therapy is know as 5-FU adjuvant therapy. “5-FU adjuvant therapy” refers to the combination of 5-FU with other treatments, such as without limitation, radiation, methyl-CCNU, Leucovorin, Oxaliplatin, irinotecin, mitomycin, cytarabine, levamisole. Specific treatment adjuvant regimens are known in the art as FOLFOX, FOLFOX4, MOF (semustine (methyl-CCNU), vincrisine (Oncovin) and 5-FU).

For a review of these therapies see Beaven and Goldberg (2006) Oncology 20(5):461-460. An example of such is an effective amount of 5-FU and Leucovorin. Other chemtherapeutics can be added, e.g., Oxaliplatin.

“5-Fluorouracil” or “5-FU” is a pyrimidine analog and an antimetabolite chemotherapeutic anticancer agent. It has been in use against cancer for about 40 years, acts in several ways, but principally as a thymidylate synthase inhibitor, interrupting the action of an enzyme which is a critical factor in the synthesis of pyrimidine-which is important in DNA replication. It finds use particularly in the treatment of colorectal cancer and pancreatic cancer.

Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

“Oxaliplatin” (Eloxatin®) is a platinum-based chemotherapy drug in the same family as cisplatin and carboplatin. It is typically administered in combination with fluorouracil and Leucovorin in a combination known as FOLFOX for the treatment of colorectal cancer. Compared to cisplatin the two amine groups are replaced by cyclohexyldiamine for improved antitumour activity. The chlorine groups are replaced by the oxalato bidentate derived from oxalic acid in order to improve water solubility. Equivalents to Oxaliplatin are known in the art and include without limitation cisplatin, carboplatin, aroplatin, lobaplatin, nedaplatin, and JM-216 (see McKeage et al. (1997) J. Clin. Oncol. 15:2691-2700 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

Leucovorin or folinic acid, the active form of folic acid in the body. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid /V[4 [[(2amino-5-formyl1,4,5,6,7,8hexahydro4oxo6-pteridinyl)methyl]amino]benzoyl], calcium salt (1:1).

“FOLFOX” is an abbreviation for a type of combination therapy that is used to treat colorectal cancer. In includes 5-FU, Oxaliplatin and Leucovorin. Information regarding this treatment is available on the National Cancer Institute's web site, cancer.gov, last accessed on Jan. 16, 2008.

### Descriptive Embodiments

This invention provides a method for selecting a therapeutic regimen or determining if a certain therapeutic regimen is more likely to treat a malignant condition such as cancer or is the appropriate chemotherapy for that patient than other available chemotherapies. In general, a therapy is considered to “treat” cancer if it provides one or more of the following treatment outcomes: reduce or delay recurrence of the cancer after the initial therapy; time to tumor progression (TTP), longer or enhanced time to tumor recurrence (TTR), decrease in tumor load or size (tumor response or TR), increase median survival time (OS) or decrease metastases. The method is suited to determining which patients will be responsive or experience a positive treatment outcome to pyrimidine based antimetabolites and efficacy enhancing agents chemotherapy or an equivalent of such therapy. These methods are useful to select therapies for highly aggressive cancers such as colon cancer or metastatic colon cancer.

For the practice of the method, the gastrointestinal or lung cancer is a metastatic or non-metastatic cancer selected from the group consisting of rectal cancer, colorectal cancer, colon cancer, gastric cancer, lung cancer, non-small cell lung cancer and esophageal cancer. In one embodiment, the patient is suffering from colorectal or colon cancer and in a further embodiment, is suffering from metastatic colorectal or colon cancer. In a further aspect, the patient is a stage II or stage III colon cancer patient. Without being bound by theory, Applicants intend that the methods are also useful to identify patients likely to respond to the combination therapy when the patient is suffering from lung cancer, ovarian cancer, esophageal, head and neck cancer or hepatocarcinoma as these cancers have been successfully treated with an effective amount of a pyrimidine-based antimetabolite chemotherapy drug and a platinum based chemotherapy drug such as 5-FU and/or Oxaliplatin and equivalents of each thereof alone or in combination with other inert carriers of no therapeutic significance to the combination. In a further aspect, an effective amount of a further therapy is administered such as an effective amount of Leucovorin.

The therapy that the patient is likely responsive to is a chemotherapy comprising, or alternatively consisting essentially of, or alternatively consisting of, administration of an effective amount of a pyrimidine-based antimetabolite chemotherapy drug such as 5-fluorouracil or an equivalent thereof and an adjuvant or efficacy enhancing agent. A platinum-based chemotherapy drug such as Oxaliplatin or an equivalent thereof can be added to the treatment. FOLFOX is an example of a combination chemotherapy comprising administration of 5-fluorouracil, Leucovorin, and Oxaliplatin.

Patient samples can include a lung or gastrointestinal or other noted tumor cell or tissue sample, or normal tissue such as peripheral blood lymphocytes. In one aspect, the suitable cell or tissue sample comprises a colorectal cancer cell or tissue sample.

In one embodiment, the therapy further comprises adjuvant radiation therapy, lymphectomy, surgical removal of the tumor or other suitable therapy.

The method comprises screening for a genomic polymorphism or genotype identified in Tables 1 and 2, above.

Methods to identify the polymorphisms identified in Tables 1 and 2 above are known in the art. For example, the VEGF allele with 936C/T polymorphism (also identified as VEGF +936 or VEGF C+936T herein) is identified and described in Zhang et al. (2006) Pharmacogenet. Genomics 7:475-483 and methods for identification are taught in U.S. Patent Publ. No. 2006/0115827. The VEGFR2 (KDR) allele at position 4422 with the CA repeat polymorphism is identified and 80 described in Kariyazono et al. (2004) Pediatr. Res. 56: 953-959. The IL-6 allele with the polymorphism 174G/C, IL-8 allele with polymorphism -251T/A, CXCR1 allele with polymorphism 2607G/C, and MMP9 allele with polymorphism −1562C/T, are identified and described in U.S. Patent Publ. No. 2006/0115827. The EGFR allele with the CA repeat polymorphisms at position 496 are identified and described in Lakin et al. (2004) Caner Treat. Rev. 30:1-17 and Gebhardt et al. (1999) J. Biol. Chem. 274:13176-13180. The VEGF allele with polymorphism 634 G/C is identified and described in Sfar (2006) 35(1-2):21-28. The IL-1β allele with polymorphism 3954 C/T is identified and described in Voronov et al. (2003) Proc. Natl. Acad. Sci. USA 100(5):2645-2650 and Pociot et al. (1992) Eur. J. Clin. Invest. 22(6):396-402. The EGF genotype 61 A/G is described in Goto et al., (2005) Cancer Epidemiol. Biomarkers Prey. 14:2454-2456. The ARNT allele with polymorphism G/C in exon 8 are identified and described in Scheel et al (2002) J. Hum. Genet. 47(5):217-224. The Hif-1α polymorphism 1772 C/T is identified and described in Tanimoto et al. (2003) Carcinogenesis 24(11):1779-1783. The TGF-β allele polymorphism 29T/C is identified and described in Brazova et al. (2006) Clin. Immunol. 121(3):350-357. The NRP-1 allele 3′ end C/T polymorphism is identified and described in U.S. Patent Publ. No. 2005/0244834. The Leptin allele with the polymorphism -2548 G/A is identified and described in Hoffstedt et al. (2002) Horm. Metab. Res. 34:355-359. The AM allele with the 3′ end CA repeat polymorphism is identified and described in Ishimitsu et al. (2001) Hypertension 38:9-12. The PLGF allele is identified and described in DiSalvo et al. (1995) J. Biol. Chem. 270(13):7717-7723. The CXCR2 allele with the polymorphism 785 C/T is identified and described in Matheson et al. (2006) H. Hum. Genet. 51:196-203. The IGF2 allele polymorphism 4205 G/A is identified and described in Kaur et al. (2005) Tumour Biol. 26(3):147-152. The FGFR4 allele with the polymorphism 388 G/A is identified and described in Stret et al. (2006) Br. J. Cancer 94:1879-1886. The IGFBP allele with the polymorphism 2133 G/C is identified and described in Le Marchand et al. (2005) Cancer Epidemiol. Biomarkers Prey. 14(5):1319-1321. Methods for identification of the Cox-2 polymorphism G765C are described in Pereira et al. (2006) World J. Gastroenterol 12:5473-5478. The polymorphism in E-cadherin (−160C/A) is identified as well as methods for it's detection are known in the art and reported in U.S. Patent Publications Nos. 2006/0094012 and 2006/0115827. The TF allele with the polymorphism −603 A/G is identified and described in Reny et al (2004) Thromb. Haemost. 91:248-254. The MMP2 allele polymorphism −1306C/T is identified and described in Grieu et al. (2004) Breast Cancer Res. Treat. 88(3):197-204. The MMP7 allele polymorphism −181 A/G is identified and described in Wang et al. (2005) Int. J. Cancer 114:19-31. The ICAM allele with the polymorphism 241 G/A is identified and described in Howell et al. (2005) Int. J. Immunogenet. 32(6):367-373. The MDM-2 allele with the polymorphism 309 T/G is identified and described in Onat et al. (2006) Anticancer Res. 26(5A):100-105. The GLUT-1 allele is identified and described in Wang et al. (2005) Ann Neurol. 57(1):111-118. The LDH-5 allele with the polymorphism Exon 5 C/T or G/A are identified and described in Koukourakis et al. (2006) Lung Cancer 53(3):257-262. The SDF1 allele with the polymorphism 3′UTR G/A is identified and described in Gerli et al. (2005) Clin. Chem. 51(12):2411-2414. The Survivin allele with the polymorphism 31 G/C is identified and described in Borbely et al. (2007) J. Clin. Pathol. 60(3):303-306. The ADAM10 allele with the polymorphism in the 5′UTR G/A is identified and described in Wollmer et al. (2002)

Psychiatr Genet. 12(3):155-160. The ADAM17 allele is identified and described in Mochizuki and Okada (2007) Cancer Sci. 98(5):621-628.

Diagnostic Methods

The invention further provides diagnostic methods, which are based, at least in part, on determination of the identity of the polymorphic region of the alleles identified in Tables 1 and 2, above.

For example, information obtained using the diagnostic assays described herein is useful for determining if a subject will likely respond to cancer treatment of a given type. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. Expression patterns of individual patients can then be compared to the expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Detection of point mutations or additional base pair repeats can be accomplished by molecular cloning of the specified allele and subsequent sequencing of that allele using techniques known in the art, in some aspects, after isolation of a suitable nucleic acid sample using methods known in the art. Alternatively, the gene sequences can be amplified directly from a genomic DNA preparation from the tumor tissue using PCR, and the sequence composition is determined from the amplified product. As described more fully below, numerous methods are available for isolating and analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

In other detection methods, it is necessary to first amplify at least a portion of the gene of interest prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad. Sci, USA 74:560) or Sanger et al. (1977) Proc. Nat. Acad. Sci, 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO 94/16101, entitled DNA Sequencing by Mass Spectrometry by Koster; U.S. Pat. No. 5,547,835 and international patent application Publication Number WO 94/21822 entitled “DNA Sequencing by Mass Spectrometry Via Exonuclease Degradation” by Koster; U.S. Pat. No. 5,605,798 and International Patent Application No. PCT/US96/03651 entitled DNA Diagnostics Based on Mass Spectrometry by Koster; Cohen et al. (1996) Adv. Chromat. 36:127-162; and Griffin et al. (1993) Appl. Biochem. Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S lnuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility is used to identify the particular allelic variant. For example, single strand conformation polymorphism (SSCP) may be used to detect differences in electrophoretic mobility between mutant and wild type nucleic acids (Orita et al. (1989) Proc. Natl. Acad. Sci USA 86:2766; Cotton (1993) Mutat. Res. 285:125-144 and Hayashi (1992) Genet Anal Tech. Appl. 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polylmorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. supra, is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al. (1989) Nucl. Acids. Res. 17:7779-7784; Sokolov, B. P. (1990) Nucl. Acids Res. 18:3671; Syvanen, A.-C. et al. (1990) Genomics 8:684-692; Kuppuswamy, M. N. et al. (1991) Proc. Natl. Acad. Sci. (U.S.A.) 88:1143-1147; Prezant, T. R. et al. (1992) Hum. Mutat. 1:159-164; Ugozzoli, L. et al. (1992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C. et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

Often a solid phase support or carrier is used as a support capable of binding of a primer, probe, polynucleotide, an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject is likely responsive to the therapy as described herein or has or is at risk of developing disease such as colorectal cancer.

Sample nucleic acid for use in the above-described diagnostic and prognostic methods can be obtained from any suitable cell type or tissue of a subject. For example, a subject's bodily fluid (e.g. blood) can be obtained by known techniques (e.g., venipuncture). Alternatively, nucleic acid tests can be performed on dry samples (e.g., hair or skin). Fetal nucleic acid samples can be obtained from maternal blood as described in International Patent Application No. WO91/07660 to Bianchi. Alternatively, amniocytes or chorionic villi can be obtained for performing prenatal testing.

Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) PCR IN SITU HYBRIDIZATION: PROTOCOLS AND APPLICATIONS, Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

The invention further provides methods for detecting the single nucleotide polymorphism in the gene of interest. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site. Cohen et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet et al. supra, is preferably a heterogeneous phase assay, in which the primer or the target molecule is immobilized to a solid phase.

Recently, several primer-guided nucleotide incorporation procedures for assaying polymorphic sites in DNA have been described (Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

Antibodies directed against wild type or mutant peptides encoded by the allelic variants of the gene of interest may also be used in disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook and Russell (2000) supra. The protein detection and isolation methods employed herein can also be such as those described in Harlow and Lane, (1999) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject has or is at risk of developing disease such as colorectal cancer.

In one particular aspect, the invention provides a prognostic panel of genetic markers comprising a primer or nucleic acid probe that identifies the genotype of a patient sample for at least one or more genetic polymorphism of the group: VEGF at nt 936 C/T; IL-8 at nt −251 T/A; VEGFR2 (KDR) at position 4422; the number of 20 CA repeats for EGFR at 496 in Intron I; the number of alleles with <14 CA repeats for AM 3′UTR CA repeat; IL-1β at nt 3954 C/T or VEGF at nt 936 C/T and IL-8 at nt −251 T/A. In another embodiment, the panel comprises probes or primers are attached to a microarray specific to identify the polymorphisms. These may in one aspect be detectably labeled. In a yet further aspect, the panel identifies the genotype of a plurality of polymorphisms selected from the group consisting of: at least two, at least three, at least four, at least five, at least six and all seven of the genetic polymorphisms.

In one embodiment, the the panel determines whether a gastrointestinal cancer or lung cancer patient in need thereof will likely respond to a therapy comprising the administration of an effective amount of a pyrimidine based antimetabolite and an effective amount of an efficacy enhancing agent. In another embodiment, the panel determines whether a gastrointestinal cancer or lung cancer patient in need of additional therapy is most likely to benefit from pyrimidine based antimetabolite and efficacy enhancing agent based chemotherapy.

Sample nucleic acid for use in the above-described diagnostic and prognostic methods can be obtained from any cell type or tissue of a subject. For example, a subject's bodily fluid (e.g. blood) can be obtained by known techniques (e.g., venipuncture). Alternatively, nucleic acid tests can be performed on dry samples (e.g., hair or skin) Fetal nucleic acid samples can be obtained from maternal blood as described in International Patent Application No. WO 91/07660 to Bianchi. Alternatively, amniocytes or chorionic villi can be obtained for performing prenatal testing.

Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo (1992) “PCR In Situ Hybridization: Protocols And Applications,” Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

The invention described herein relates to methods and compositions for determining and identifying the allele present at the gene of interest's locus. This information is useful to diagnose and prognose disease progression as well as select the most effective treatment among treatment options. Probes can be used to directly determine the genotype of the sample or can be used simultaneously with or subsequent to amplification. The term “probes” includes naturally occurring or recombinant single- or double-stranded nucleic acids or chemically synthesized nucleic acids. They may be labeled by nick translation, Klenow fill-in reaction, PCR or other methods known in the art. Probes of the present invention, their preparation and/or labeling are described in Sambrook and Russell (2000) supra. A probe can be a polynucleotide of any length suitable for selective hybridization to a nucleic acid containing a polymorphic region of the invention. Length of the probe used will depend, in part, on the nature of the assay used and the hybridization conditions employed.

In one embodiment of the invention, probes are labeled with two fluorescent dye molecules to form so-called “molecular beacons” (Tyagi and Kramer (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis (1998) Science 279:1228-9) as has the use of multiple beacons simultaneously (Marras (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a polymorphism. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule—quencher molecule pair that specifically anneals to a region of a target polynucleotide containing the polymorphism.

Probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarry” and similar technologies are know in the art. Examples of such include, but are not limited to LabCard (ACLARA Bio Sciences Inc.); GeneChip (Affymetric, Inc.); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarrying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or a “microarray” are also described in US Patent Publ. Nos.: 2007-0111322, 2007-0099198, 2007-0084997, 2007-0059769 and 2007-0059765 and U.S. Pat. Nos. 7,138,506, 7,070,740, and 6,989,267.

In one aspect, “gene chips” or “microarrays” containing probes or primers of Tables 1 and 2 alone or in combination are prepared. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genotypes of the patient is then determined with the aid of the aforementioned apparatus and methods.

Nucleic Acids

In one aspect, the nucleic acid sequences of the gene's allelic variants, or portions thereof, can be the basis for probes or primers, e.g., in methods for determining the identity of the allelic variant of a polymorphic region of interest, e.g., VEGF 936 C/T. Thus, they can be used in the methods of the invention to determine which therapy is most likely to treat an individual's cancer.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers for use in the methods of the invention are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the invention are nucleic acids which hybridize to the region of interest and which are not further extended. For example, a probe is a nucleic acid which hybridizes to the polymorphic region of the gene of interest, and which by hybridization or absence of hybridization to the DNA of a subject will be indicative of the identity of the allelic variant of the polymorphic region of the gene of interest.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids which are capable of selectively hybridizing to an allelic variant of a polymorphic region of the gene of interest. Thus, such primers can be specific for the gene of interest sequence, so long as they have a nucleotide sequence which is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al., (1989) Proc. Natl. Acad. Sci. U.S.A. 86:6553-6556; Lemaitre et al., (1987) Proc. Natl. Acad. Sci. 84:648-652; and PCT Publication No. WO 88/09810, published Dec. 15, 1988), hybridization-triggered cleavage agents, (see, e.g., Krol et al., (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook and Russell (2000) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

Methods of Treatment

The invention further provides methods of treating subjects having solid malignant tissue mass or tumor as identified above, which includes for example, rectal cancer, colorectal cancer, (including metastatic CRC), colon cancer (metastatic colon cancer), gastric cancer, lung cancer (including non-small cell lung cancer) and esophageal cancer. In one embodiment, the method comprises, or alternatively consists essentially of or yet further consists of: (a) determining the identity of the allelic variant as identified herein; and (b) administering to the subject an effective amount of a compound or therapy (e.g., pyrimidine based antimetabolites and efficacy enhancing agents or chemical equivalent thereof). This therapy can be combined with other suitable therapies or treatments such as radiation therapy.

In certain embodiments, an effective amount of Fluorouracil (5-FU) or a chemical equivalent and an efficacy enhancing agent are administered to the patient. In general, compositions comprising these compounds can be prepared in accordance with known formulation techniques to provide a composition suitable for oral, topical, transdermal, rectal, inhalation, or parenteral (intravenous, intramuscular, or intraperitoneal) administration, and the like. In general the dosing, route of administration, and administration schedule of this compound is well know in the art. Examples of such can be found in, but are not limited to Gramont et al. (2000) J. Clin. Oncol. 18(16):2938-2947 and Cassidy et al. (2004) J. Clin. Oncol. 22(11):2084-2091.

Fluorouracil (5-FU) or a chemical equivalent alone or with an adjuvant is administered in a therapeutically effective amount sufficient to treat cancer in a subject and may contain from about 1.0 to 2000 mg/m²/day of compound, for example about 1, 5, 10, 15, 20, 25, 50, 75, 100, 125, 150, 175, 200, 225, 250, 275, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, to 2000 mg/m².

Fluorouracil (5-FU) or a chemical equivalent alone or an adjuvant may be administered parenterally, e.g., intravenously, intramuscularly, intravenously, subcutaneously, or interperitonically. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of Fluorouracil (5-FU) or a chemical equivalent and an adjuvant may be prepared in suitable diluents such as water, ethanol, glycerol, liquid polyethylene glycol(s), various oils, and/or mixtures thereof, and others known to those skilled in the art.

The pharmaceutical forms of Fluorouracil (5-FU) or a chemical equivalent suitable or an adjuvant for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In one aspect of the invention, a chemical equivalent of 5-FU (a pyrimidine based anti-metabolite) selected from the group of, but not limited to Cytarabine and Gemcitabine as described in Maring et al. (2005) Pharmacogenomics J. 5(4):226-243; and Floxuridine as described in Mayer (1992) Cancer. 70(5 Suppl):1414-1424, can be to treat patients identified as having the appropriate genetic polymorphisms.

In certain embodiments, an effective amount of Leucovorin (Folinic acid) or a chemical equivalent as an adjuvant is administered to the patient for the purpose of enhancing the cytotoxic effects of 5-FU or a chemical equivalent. In general, compositions comprising these compounds can be prepared in accordance with known formulation techniques to provide a composition suitable for oral, topical, transdermal, rectal, inhalation, or parenteral (intravenous, intramuscular, or intraperitoneal) administration, and the like. In general the dosing, route of administration, and administration schedule of this compound is well know in the art. Examples of such can be found in, but are not limited to Gramont et al. (2000) J. Clin. Oncol. 18(16):2938-2947 and Cassidy et al. (2004) J. Clin. Oncol. 22(11):2084-2091.

Leucovorin or a chemical equivalent is administered in a therapeutically effective amount sufficient to increase the effectiveness of 5-FU or a chemical equivalent to treat cancer in a subject and may contain from about 1.0 to 1000 mg/m²/day of compound, for example about 1, 5, 10, 15, 20, 25, 50, 75, 100, 125, 150, 175, 200, 225, 250, 275, 300, 325, 350, 375, 400, 425, 450, 475, 500, 550, 600, 650, 700, 750, 800, 850, 900, 950, to 1000 mg/m².

Leucovorin or a chemical equivalent may be administered parenterally, e.g., intravenously, intramuscularly, intravenously, subcutaneously, or interperitonically. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of Leucovorin or a chemical equivalent may be prepared in suitable diluents such as water, ethanol, glycerol, liquid polyethylene glycol(s), various oils, and/or mixtures thereof, and others known to those skilled in the art.

The pharmaceutical forms of Leucovorin or a chemical equivalent suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In certain embodiments, an effective amount of Oxaliplatin or a chemical equivalent is administered to the patient as an enhancing agent or adjuvant. In general, compositions comprising these compounds can be prepared in accordance with known formulation techniques to provide a composition suitable for oral, topical, transdermal, rectal, inhalation, or parenteral (intravenous, intramuscular, or intraperitoneal) administration, and the like. In general the dosing, route of administration, and administration schedule of this compound is well know in the art. Examples of such can be found in, but are not limited to Gramont et al. (2000) J. Clin. Oncol. 18(16):2938-2947 and Cassidy et al. (2004) J. Clin. Oncol. 22(11):2084-2091.

Oxaliplatin or a chemical equivalent is administered in a therapeutically effective amount sufficient to treat cancer in a subject and may contain from about 1.0 to 2000 mg/m²/day of compound, for example about 1, 5, 10, 15, 20, 25, 50, 75, 100, 125, 150, 175, 200, 225, 250, 275, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, to 2000 mg/m².

Oxaliplatin or a chemical equivalent may be administered parenterally, e.g., intravenously, intramuscularly, intravenously, subcutaneously, or interperitonically. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of Oxaliplatin or a chemical equivalent may be prepared in suitable diluents such as water, ethanol, glycerol, liquid polyethylene glycol(s), various oils, and/or mixtures thereof, and others known to those skilled in the art.

The pharmaceutical forms of Oxaliplatin or a chemical equivalent suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In one aspect of the invention, a chemical equivalent of Oxaliplatin (a platinum based alkylating agent) selected from the group of, but not limited to Carboplatin and Cisplatin as described in Galanski and Keppler (2007) Anticancer Agents Med. Chem. 7(1):55-73; and BBR3464 as described in Boulikas and Vaugiouka (2003) Oncol. Rep. 10(6):1663-1682, can be used in combination with pyrimidine based antimetabolite and efficacy enhancing agent based chemotherapy to treat patients identified as having the appropriate genetic polymorphism.

In certain embodiments, an effective amount of a pyrimidine based antmetabolite and a platinum-based alkylating agent in adjuvant chemotherapy including, but are not limited to Fluorouracil (5-FU), Leucovorin, and Oxaliplatin (FOLFOX) or their chemical equivalents are co-administered to the patient. In general the dosing, route of administration, and administration schedule of these compounds are well know in the art. Examples of such can be found in, but are not limited to Gramont et al. (2000) J. Clin. Oncol. 18(16):2938-2947 and Cassidy et al. (2004) J. Clin. Oncol. 22(11):2084-2091.

In certain embodiments, an effective amount of Irinotecan or a chemical equivalent is administered to the patient as an enhancing agent or adjuvant. Compositions comprising these compounds can be prepared in accordance with known formulation techniques to provide a composition suitable for oral, topical, transdermal, rectal, inhalation, or parenteral (intravenous, intramuscular, or intraperitoneal) administration, and the like. Detailed guidance for preparing compositions of the invention are found by reference to the 18^{th} or 19^{th} Edition of Remington's Pharmaceutical Sciences, Published by the Mack Publishing Co., Easton, Pa. 18040.

Irinotecan or a chemical equivalent is administered in a therapeutically effective amount sufficient to treat cancer in a subject and may contain from about 1.0 to 1000 mg of compound, for example about 1, 5, 10, 15, 20, 25, 50, 75, 100, 125, 150, 175, 200, 225, 250, 275, 300, 325, 350, 375, 400, 425, 450, 475, to 500 mg.

Irinotecan or a chemical equivalent can be administered orally in a suitable formulation as an ingestible tablet, a buccal tablet, capsule, caplet, elixir, suspension, syrup, trouche, wafer, lozenge, and the like. Generally, the most straightforward formulation is a tablet or capsule (individually or collectively designated as an “oral dosage unit”). Suitable formulations are prepared in accordance with a standard formulating techniques available that match the characteristics of the compound to the excipients available for formulating an appropriate composition. A tablet or capsule will contain about 50 to about 500 mg.

Irinotecan or a chemical equivalent may deliver the compound rapidly or may be a sustained-release preparation. The compound may be enclosed in a hard or soft capsule, may be compressed into tablets, or may be incorporated with beverages, food or otherwise into the diet. The percentage of the final composition and the preparations may, of course, be varied and may conveniently range between 1 and 90% of the weight of the final form, e.g., tablet. The amount in such therapeutically useful compositions is such that a suitable dosage will be obtained. An alternative composition according to the current invention are prepared so that an oral dosage unit form contains between about 5 to about 50% by weight (% w) in dosage units weighing between 50 and 1000 mg.

The suitable formulation of an oral dosage unit of Irinotecan or a chemical equivalent may also contain: a binder, such as gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other material may be present as coating or to otherwise modify the physical form of the oral dosage unit. The oral dosage unit may be coated with shellac, a sugar or both. Syrup or elixir may contain the compound, sucrose as a sweetening agent, methyl and propylparabens as a preservative, a dye and flavoring. Any material utilized should be pharmaceutically-acceptable and substantially non-toxic. Details of the types of excipients useful may be found in the nineteenth edition of “Remington: The Science and Practice of Pharmacy,” Mack Printing Company, Easton, Pa. See particularly chapters 91-93 for a fuller discussion.

Irinotecan or a chemical equivalent may be administered parenterally, e.g., intravenously, intramuscularly, intravenously, subcutaneously, or interperitonically. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of Irinotecan or a chemical equivalent may be prepared in suitable diluents such as water, ethanol, glycerol, liquid polyethylene glycol(s), various oils, and/or mixtures thereof, and others known to those skilled in the art.

The pharmaceutical forms of Irinotecan or a chemical equivalent suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

Usefully, Irinotecan or a chemical equivalent of the invention is solubilized in liposomes. The liposomes may include, for example, lipids such as cholesterol, phospholipids, or micelles comprised of surfactant such as, for example, sodium dodecylsulfate, octylphenolpolyoxyethylene glycol, or sorbitan mono-oleate. Typically, the compound of the invention binds to the lipid bilayer membrane of the liposome with high affinity. The liposome bound prodrug can preferably intercalate between the acyl chains of the lipid. The lactone ring of the camptothecin-derivative, membrane-bound compound of the invention is thereby removed from the aqueous environment inside and outside of the liposome and further protected from hydrolysis. Since the liposome-bound drug is protected from hydrolysis, the antitumor activity of the drug is preserved. If Irinotecan or a chemical equivalent of the invention has a lower affinity for the liposome membrane and thus disassociates from the liposome membrane to reside in the interior of liposome, the pH of the interior of the liposomes may be reduced thereby preventing hydrolysis of such compound of the invention.

U.S. Pat. No. 6,096,336 provides further guidance for preparing liposomal compositions useful in this invention.

In one aspect of the invention, a chemical equivalent of Irinotecan (a topoisomerase I inhibitor) selected from the group of, but not limited to, Campothecine derivatives including CPT-11/Irinotecan, SN-38, APC, NPC, camptothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as decribed in Pommier Y. (2006) Nat. Rev. Cancer 6(10):789-802 and US Patent Publ. No. 2005/0250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11(8):1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-125, Crow et al. (1994) J. Med. Chem. 37(19):3191-3194, and (Crespi et al. (1986) Biochem. Biophys. Res. Commun. 136(2):521-8, can be used in combination therapy with the antibody based chemotherapy described above to treat patients identified as having the appropriate genetic markers.

In another aspect of the invention, dual topoisomerase I and II inhibitors selected from the group of, but not limited to, Saintopin and other Naphthecenediones, DACA and other Acridine-4-Carboxamindes, Intoplicine and other Benzopyridoindoles, TAS-103 and other 7H-indeno[2,1-c]Quinoline-7-ones, Pyrazoloacridine, XR 11576 and other Benzophenazines, XR 5944 and other Dimeric compounds, 7-Oxo-7H-dibenz[f,ij]Isoquinolines and 7-oxo-7H-benzo[e]Perimidines, and Anthracenyl-amino Acid Conjugates as described in Denny and Baguley (2003) Curr. Top. Med. Chem. 3(3):339-353, can be used in combination with pyrimidine based antimetabolite and efficacy enhancing agent based chemotherapy to treat patients identified as having the appropriate genetic polymorphism.

The agents identified herein as effective for their intended purpose can be administered to subjects or individuals identified by the methods herein as suitable for the therapy, Therapeutic amounts can be empirically determined and will vary with the pathology being treated, the subject being treated and the efficacy and toxicity of the agent.

This invention also provides the use of any one or more of the above-described compositions in the preparation of a medicament to treat a patient as identified herein as likely responsive to the administration of this therapy.

Kits

As set forth herein, the invention provides diagnostic methods for determining the type of allelic variant of a polymorphic region present in the gene of interest or the expression level of a gene of interest. In some embodiments, the methods use probes or primers comprising nucleotide sequences which are complementary to the polymorphic region of the gene of interest. Accordingly, the invention provides kits for performing these methods as well as instructions for carrying out the methods of this invention such as collecting tissue and/or performing the screen, and/or analyzing the results, and/or administration of an effective amount of the pyrimidine based chemotherapy alone or in combination with efficacy enhancing agent or radiation, such as 5-FU or in combination with Oxaliplatin.

In an embodiment, the invention provides a kit for determining whether a subject is likely responsive to cancer treatment or alternatively one of various treatment options. The kits contain one of more of the compositions described above and instructions for use. As an example only, the invention also provides kits for determining response to cancer treatment containing a first and a second oligonucleotide specific for the polymorphic region of the gene. Oligonucleotides “specific for” a genetic locus bind either to the polymorphic region of the locus or bind adjacent to the polymorphic region of the locus. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, and preferably less than 1 kb from the polymorphism. Specific oligonucleotides are capable of hybridizing to a sequence, and under suitable conditions will not bind to a sequence differing by a single nucleotide.

Accordingly, the invention provides kits for performing these methods.

In an embodiment, the invention provides a kit for determining whether a subject responds to cancer treatment or alternatively one of various treatment options. The kits contain one of more of the compositions described above and instructions for use. As an example only, the invention also provides kits for determining response to cancer treatment containing a first and a second oligonucleotide specific for the polymorphic region of the gene.

Oligonucleotides “specific for” a genetic locus bind either to the polymorphic region of the locus or bind adjacent to the polymorphic region of the locus. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, and preferably less than 1 kb from the polymorphism. Specific oligonucleotides are capable of hybridizing to a sequence, and under suitable conditions will not bind to a sequence differing by a single nucleotide.

The kit can comprise at least one probe or primer which is capable of specifically hybridizing to the polymorphic region of the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) “AN INTRODUCTION TO RADIOIMMUNOASSAY AND RELATED TECHNIQUES” Elsevier Science Publishers, Amsterdam, The Netherlands; Bullock et al., “TECHNIQUES IN IMMUNOCYTOCHEMISTRY” Academic Press, Orlando, Fla. Vol. 1 (1982), Vol. 2 (1983), Vol. 3 (1985); Tijssen (1985) “PRACTICE AND THEORY OF IMMUNOASSAYS: LABORATORY TECHNIQUES IN BIOCHEMISTRY AND MOLECULAR BIOLOGY”, Elsevier Science Publishers, Amsterdam, The Netherlands.

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

Other Uses for the Nucleic Acids of the Invention

The identification of the allele of the gene of interest can also be useful for identifying an individual among other individuals from the same species. For example, DNA sequences can be used as a fingerprint for detection of different individuals within the same species. Thompson, J. S. and Thompson, eds., (1991) “Genetics in Medicine”, W B Saunders Co., Philadelphia, Pa. This is useful, e.g., in forensic studies.

The invention now being generally described, it will be more readily understood by reference to the following examples which are included merely for purposes of illustration of certain aspects and embodiments of the present invention, and are not intended to limit the invention.

### Experimental Examples

### Experiment No. 1

For the purpose of illustration only, peripheral blood sample can be collected from each patient, and genomic DNA can be extracted from white blood cells using the QiaAmp kit (Qiagen, Valencia, Calif.).

Background: Colorectal cancer is the 3^{rd} most common cause of cancer in the US with 150,000 newly diagnosed patients in the year 2007. The number of deaths from colorectal cancer average 52,000 per year. The prognosis of patients with this disease depends on several parameters including stage of the disease, age, gender, and performance status of the patient, and recently the genetic profile of the patient. In this retrospective study, tests were conducted to identify a panel of prognostic genetic markers for tumor recurrence focused on tumor angiogenesis and the tumor microenvironment.

Methods: Samples were collected from 197 patients with stage II or III colon cancer who had received 5-FU based adjuvant therapy composed of 5-FU and Leucovorin, and is some cases additional Oxaliplatin or CPT-11 (Irinotecan) chemotherapy. Several clinical predictors were evaluated including Age, Histology, Stage, Chemotherapy/Radiation received, site of recurrence, last follow-up, and death. See Table 3, below. Genomic DNA was extracted from peripheral blood and genotypes were determined using PCR based RFLP, 5′end P³³ γ[ATP] labeled PCR, and direct sequencing. Polymorphisms in the genes involved in angiogenesis (EGF, EGFR, ARNT, Hif-1α, TGF-β, VEGF, VEGFR2, NRP-1, Leptin, AM, PLGF, IL-8, IL-1β, CXCR2, CXCR1, IGF2, IL6, FGFR4, IGFBP) and gene involved in the tumor microenviroment (COX-2, ICAM, E-Cadherin, TF, MDM-2, GLUT-1, LDH-5, SDF1, MMP-2, MMP-7, MMP-9, Survivin, ADAM10, ADAM17) were evaluated for predicting tumor recurrence.

TABLE 3

| | |
|---|---|
| Stage II | Stage III |
| (N = 72) | (N = 125) |

| | N | % | N | % |
|---|---|---|---|---|
| Age, y | | | | |
| Median | 60 | | 58 | |
| Range | 22-85 | | 31-87 | |
| ≦50 | 17 | 23.6 | 29 | 23.2 |
| 50 | 55 | 76.4 | 96 | 76.8 |
| Gender | | | | |
| Male | 40 | 55.6 | 75 | 60.0 |
| Female | 32 | 44.4 | 50 | 40.0 |
| Ethnicity | | | | |
| White | 42 | 58.3 | 70 | 56.0 |
| African American | 5 | 6.9 | 7 | 5.6 |
| Asian | 4 | 5.6 | 26 | 20.8 |
| Hispanic | 21 | 29.2 | 22 | 17.6 |
| T stage | | | | |
| T1-T2 | n.a. | n.a. | 15 | 12.0 |
| T3 | 63 | 87.5 | 93 | 74.4 |
| T4 | 9 | 12.5 | 13 | 10.4 |
| Tx | n.a. | n.a. | 4 | 3.2 |
| N stage | | | | |
| N0 | 72 | 100 | n.a. | n.a. |
| N1 | n.a. | n.a. | 70 | 56.0 |
| N2 | n.a. | n.a. | 55 | 44.0 |
| Adjuvant therapy | | | | |
| 5-FU/LV | 58 | 80.6 | 76 | 60.8 |
| 5-FU/LV/Oxaliplatin | 9 | 12.5 | 31 | 24.8 |
| 5-FU/LV/CPT-11 | 5 | 6.9 | 18 | 14.4 |
| Differentiation | | | | |
| Well | 3 | 4.4 | 4 | 3.5 |
| Moderate | 52 | 76.5 | 70 | 62.0 |
| Moderate/Poor | 13 | 19.1 | 39 | 34.5 |

Results: Polymorphisms in VEGF (936 C/T; p=0.0035) and IL-8 (−251 T/A; p=0.048) were associated risk of tumor recurrence in stage III colon cancer patients (n=125). Polymorphisms in VEGFR2 (KDR) (4422 AC repeat; p=0.0037), EGFR (496 CA repeats;

0.016), AM (3′UTR CA repeat; p=0.043), and IL-1β (3954 C/T; 0.0015) were associated with tumor recurrence in stage II colon cancer patients (n=72).

Experiment No. 2

In a follow-up to the initial study reported as Experiment No. 1 above, the following study was designed and implemented as set forth below.

Methods and Patients: One hundred and twenty five patients with stage III colon cancer who were treated with 5-FU-based adjuvant chemotherapy at the University of Southern California/Norris Comprehensive Cancer Center (USC/NCCC) or the Los Angeles County/University of Southern California Medical Center (LAC/USCMC), between 1992 and 2007, were eligible for the present study. This study was conducted at the USC/NCCC and approved by the Institutional Review Board (IRB) of the University of Southern California for Medical Sciences. Patient data were collected retrospectively through chart review. Informed consent was signed by all patients involved in the study. Detailed clinic-pathologic characteristics are shown in Table 4.

TABLE 4
Demographic and Clinico-Pathologic Characteristics and Time to Tumor
Recurrence in Patients with Stage III Colon Cancer

| | | Median time to | | | | | |
|---|---|---|---|---|---|---|---|
| | | recurrence | | | | | |
| | | (TTR) yrs | | Relative risk | | Probability ± SE* | |
| | n | (95% CI) | | (95% CI) | | of 3-year recurrence | P value† |
| Age, years | | | | | | | 0.69 |
| ≦50 | 29 (23.2%) | 6.8+. | (2.3, 6.8+) | 1 | (Reference) | 0.46 ± 0.10 | |
| >50 | 96 (76.8%) | 5.2 | (2.4, 11.1) | 1.14 | (0.60, 2.15) | 0.45 ± 0.05 | |
| Sex | | | | | | | 0.63 |
| Male | 75 (60.0%) | 5.2 | (2.0, 11.1) | 1 | (Reference) | 0.45 ± 0.06 | |
| Female | 50 (40.0%) | 5.7 | (2.4, 10.4+) | 0.88 | (0.52, 1.49) | 0.45 ± 0.07 | |
| Race | | | | | | | 0.12 |
| White | 70 (56.0%) | 3.4 | (1.8, 11.1) | 1 | (Reference) | 0.47 ± 0.06 | |
| African- | 7 (5.6%) | 2.3 | (0.5, 3.3+) | 2.04 | (0.79, 5.24) | 0.79 ± 0.18 | |
| American | | | | | | | |
| Asian | 26 (20.8%) | 7.1 | (1.5, 7.7+) | 0.83 | (0.42, 1.64) | 0.43 ± 0.10 | |
| Hispanic | 22 (17.6%) | 10.4+ | (3.9, 10.4+) | 0.52 | (0.22, 1.23) | 0.28 ± 0.11 | |
| T stage | | | | | | | 0.24 |
| T1‡ | 2 (1.6%) | | | | | | |
| T2‡ | 13 (10.4%) | 7.4+ | (7.4+) | 1 | (Reference) | 0.23 ± 0.12 | |
| T3 | 93 (74.4%) | 3.9 | (2.3, 11.1) | 3.02 | (0.94, 9.72) | 0.47 ± 0.06 | |
| T4 | 13 (10.4%) | 2.0 | (1.0, 10.7+) | 3.55 | (0.92, 13.71) | 0.57 ± 0.14 | |
| Tx | 4 (3.2%) | 2.7 | (1.3, 11.3+) | 3.10 | (0.61, 15.66) | 0.50 ± 0.25 | |
| N stage | | | | | | | 0.52 |
| N1 | 70 (56.0%) | 6.6 | (2.5, 11.3+) | 1 | (Reference) | 0.42 ± 0.06 | |
| N2 | 55 (44.0%) | 5.2 | (1.7, 12.4+) | 1.18 | (0.71, 1.98) | 0.48 ± 0.07 | |
| N of resected | | | | | | | 0.045 |
| lymph nodes | | | | | | | |
| <12 | 39 (31.2%) | 2.5 | (1.4, 6.6) | 1 | (Reference) | 0.56 ± 0.08 | |
| ≧12 | 86 (68.8%) | 7.1 | (2.8, 10.4+) | 0.60 | (0.36, 1.01) | 0.40 ± 0.06 | |
| Adjuvant therapy | | | | | | | 0.69 |
| 5-FU | 76 (60.8%) | 3.9 | (1.7, 12.4+) | 1 | (Reference) | 0.47 ± 0.06 | |
| 5- | 31 (24.8%) | 3.4 | (1.8, 4.2+) | 0.99 | (0.50, 1.94) | 0.49 ± 0.12 | |
| FU/LV/Oxaliplatin | | | | | | | |
| 5-FU/LV/CPT- | 18 (14.4%) | 7.1+ | (2.0, 7.1+) | 0.71 | (0.32, 1.58) | 0.37 ± 0.12 | |
| 11 | | | | | | | |
| Tumor site | | | | | | | 0.90 |
| Left | 69 (56.1%) | 5.7 | (1.8, 10.7+) | 1 | (Reference) | 0.44 ± 0.06 | |
| Right‡ | 53 (43.1%) | 3.9 | (2.0, 12.4+) | 0.97 | (0.57, 1.63) | 0.46 ± 0.07 | |
| Left and Right‡ | 1 (0.8%) | | | | | | |
| Differentiation | | | | | | | 0.34 |
| Well‡ | 4 (3.5%) | | | | | | |
| Moderate‡ | 70 (62.0%) | 6.6 | (2.6, 12.4+) | 1 | (Reference) | 0.41 ± 0.06 | |
| Moderate/Poor | 39 (34.5%) | 2.5 | (1.7, 11.1+) | 1.31 | (0.75, 2.28) | 0.51 ± 0.09 | |

*Greenwood SE,.
+Estimates were not reached.
†Based on log-rank test.
‡Grouped together for the estimates of relative risk and probability ± SE of 3-year recurrence.

Genotyping: Whole blood was collected and genomic DNA was extracted using the QIAamp kit (Qiagen, CA, USA). The majority of the samples were tested using polymerase chain reaction restriction fragment length polymorphism (PCR-RFLP) technique. Briefly, forward and reverse primers were used for PCR amplification, PCR products were digested by restriction enzymes (New England Biolab, Massachusetts, USA) and alleles were separated using a 4% NuSieve ethidium bromide stained agarose gel. Forward and reverse primer, restriction enzymes and annealing temperatures are listed in Table 5. Samples were analyzed by direct sequencing, if no matching restriction enzyme could be found.

TABLE 5
Polymorphisms of Genes in Angiogenesis and Time to Recurrence in
Patients with Stage III Colon Cancer

| | | Median time to | | | | | |
|---|---|---|---|---|---|---|---|
| | | recurrence | | | | Probability ± | |
| | | (TTR) | | Relative risk | | SE* of 3-year | |
| | n | yrs (95% CI) | | (95% CI) | | recurrence | P value† |
| VEGF C + 936T | | | | | | | 0.003 |
| C/C | 80 (66.1%) | 2.6 | (1.7, 5.7) | 1 | (Reference) | 0.54 ± 0.06 | |
| C/T‡ | 37 (30.6%) | 11.1 | (6.6, 12.4+) | 0.42 | (0.22, 0.79) | 0.25 ± 0.07 | |
| T/T‡ | 4 (3.3%) | | | | | | |
| VEGF 634 | | | | | | | 0.76 |
| G/G | 43 (35.5%) | 11.1 | (1.7, 12.4+) | 1 | (Reference) | 0.41 ± 0.08 | |
| G/C | 60 (49.6%) | 3.4 | (2.0, 11.3+) | 1.23 | (0.69, 2.21) | 0.48 ± 0.07 | |
| C/C | 18 (14.9%) | 5.7 | (1.5, 8.9+) | 1.06 | (0.46, 2.42) | 0.38 ± 0.12 | |
| VEGFR-2 (AC)ₙ | | | | | | | |
| repeat | | | | | | | |
| 11/9‡ | 1 (0.8%) | | | | | | |
| 11/10‡ | 1 (0.8%) | | | | | | |
| 11/11‡ | 59 (48.8%) | 7.1 | (2.6, 12.4+) | 1 | (Reference) | 0.38 ± 0.07 | 0.17 |
| 11/12 | 50 (41.3%) | 2.8 | (1.8, 11.1+) | 1.65 | (0.95, 2.86) | 0.50 ± 0.08 | |
| 12/12 | 10 (8.3%) | 6.6 | (0.9, 10.4+) | 1.71 | (0.65, 4.50) | 0.49 ± 0.18 | |
| NRP-1 3′UTR C/T | | | | | | | 0.26 |
| C/C | 35 (28.9%) | 7.1 | (2.8, 10.4+) | 1 | (Reference) | 0.35 ± 0.08 | |
| C/T | 51 (42.2%) | 2.5 | (1.7, 6.6) | 1.56 | (0.84, 2.88) | 0.54 ± 0.07 | |
| T/T | 35 (28.9%) | 11.1+ | (1.8, 11.1+) | 1.03 | (0.48, 2.17) | 0.38 ± 0.09 | |
| AM (CA)ₙ repeat | | | | | | | 0.82 |
| <14/<14 | 16 (13.2%) | 2.7 | (1.0, 7.7+) | 1 | (Reference) | 0.53 ± 0.13 | |
| <14/≧14 | 36 (29.8%) | 5.7 | (2.4, 11.1+) | 0.92 | (0.40, 2.13) | 0.40 ± 0.09 | |
| ≧14/≧14 | 69 (57%) | 5.2 | (2.3, 12.4+) | 0.80 | (0.37, 1.76) | 0.44 ± 0.06 | |
| IL 8 T-251A | | | | | | | 0.048 |
| T/T | 36 (29.8%) | 5.7 | (1.8, 11.3+) | 1 | (Reference) | 0.40 ± 0.09 | |
| T/A | 62 (51.2%) | 6.6 | (2.7, 12.4+) | 1.06 | (0.55, 2.06) | 0.41 ± 0.07 | |
| A/A | 23 (19%) | 2.4 | (1.0, 3.9) | 2.14 | (1.01, 4.53) | 0.58 ± 0.11 | |
| CXCR1 G + 2607C | | | | | | | 0.83 |
| G/G | 95 (79.8%) | 5.2 | (2.5, 11.3+) | 1 | (Reference) | 0.43 ± 0.05 | |
| G/C‡ | 21 (17.7%) | 11.1 | (2.4, 12.4+) | 0.93 | (0.48, 1.82) | 0.45 ± 0.11 | |
| C/C‡ | 3 (2.5%) | | | | | | |
| CXCR2 C + 785T | | | | | | | 0.53 |
| C/C | 41 (35.3%) | 2.7 | (1.6, 11.1+) | 1 | (Reference) | 0.50 ± 0.09 | |
| C/T | 45 (38.8%) | 7.1 | (2.4, 10.7+) | 0.70 | (0.37, 1.32) | 0.38 ± 0.08 | |
| T/T | 30 (25.9%) | 3.2 | (1.5, 12.4+) | 0.83 | (0.42, 1.62) | 0.45 ± 0.09 | |
| EGF A + 61G | | | | | | | 0.17 |
| A/A | 30 (24.8%) | 5.7 | (3.2, 12.4+) | 1 | (Reference) | 0.29 ± 0.09 | |
| A/G | 68 (56.2%) | 6.6 | (2.6, 11.3+) | 1.10 | (0.57, 2.12) | 0.44 ± 0.06 | |
| G/G | 23 (19%) | 1.5 | (0.9, 6.8+) | 1.91 | (0.88, 4.16) | 0.62 ± 0.11 | |
| EGFR G + 497A | | | | | | | 0.53 |
| G/G | 50 (41.3%) | 3.4 | (2.3, 11.3+) | 1 | (Reference) | 0.47 ± 0.07 | |
| G/A | 60 (49.6%) | 11.1 | (2.4, 12.4+) | 0.74 | (0.42, 1.30) | 0.43 ± 0.07 | |
| A/A | 11 (9.1%) | 5.2 | (1.7, 7.1+) | 0.98 | (0.42, 2.26) | 0.36 ± 0.15 | |
| EGFR (CA)ₙ repeat | | | | | | | 0.09 |
| Both (CA)ₙ <20 | 43 (38.7%) | 2.4 | (1.7, 6.6) | 1 | (Reference) | 0.55 ± 0.08 | |
| Any (CA)ₙ ≧20 | 68 (61.3%) | 7.1 | (3.2, 12.4+) | 0.63 | (0.36, 1.09) | 0.37 ± 0.06 | |
| ARNT PAS G/C | | | | | | | 0.91 |
| G/G | 37 (30.8%) | 2.7 | (1.7, 12.4+) | 1 | (Reference) | 0.54 ± 0.09 | |
| G/C | 62 (51.7%) | 6.6 | (2.4, 11.3+) | 0.89 | (0.49, 1.62) | 0.44 ± 0.07 | |
| C/C | 21 (17.5%) | 5.2 | (3.4, 10.4+) | 0.86 | (0.40, 1.88) | 0.30 ± 0.10 | |
| HIF-1a C + 1772T | | | | | | | 0.79 |
| C/C | 101 (84.2%) | 5.7 | (2.4, 12.4+) | 1 | (Reference) | 0.45 ± 0.05 | |
| C/T‡ | 18 (15%) | 3.9 | (2.8, 10.4+) | 0.90 | (0.43, 1.91) | 0.33 ± 0.13 | |
| T/T‡ | 1 (0.8%) | | | | | | |
| COX2 T + 8473C | | | | | | | 0.20 |
| T/T | 53 (44.9%) | 3.9 | (2.0, 11.3+) | 1 | (Reference) | 0.44 ± 0.07 | |
| T/C | 53 (44.9%) | 6.6 | (2.6, 12.4+) | 0.84 | (0.47, 1.50) | 0.36 ± 0.07 | |
| C/C | 12 (10.2%) | 1.0 | (1.0, 10.4+) | 1.74 | (0.78, 3.87) | 0.67 ± 0.14 | |

*Greenwood SE.
+Estimates were not reached.
†Based on log-rank test.
‡Grouped together for the estimates of relative risk and probability ± SE of 3-year recurrence

The dinucleotide polymorphisms (Table 6) were determined with 5′-end 33p γATP labeled PCR protocol with a few modifications. In summary, DNA templete, dNTPs, 5′-end 33p γATP labeled primer, unlabelled complementary primer, Taq Polymerase (Perkin Elmer Inc., Connecticut, USA) and PCR Buffer were used together in a final PCR. The reaction was carried out and the reaction products were separated using a 6% denaturing polyacrylamid DNA sequencing gel, which then was vacuum blotted for 1 h at 80° C. and exposed to an XAR film (Eastman-Kodak Co. New York, USA) overnight. The exact number of repeats was confirmed by direct sequencing.

TABLE 6

Primer Sequences, Annealing Temperatures, and Restriction Enzymes

| | | | | |
|---|---|---|---|---|
| Gene | Forward-Primer (5′3′) | Reverse-Primer (5′3′) | Enzyme | Annealing |

| | | | | |
|---|---|---|---|---|
| VEGF | AAGGAAGAGGAGACT | TAAATGTATGTATGTGGG | Nla III | 60° |
| C + 936T | CTGCGCAGAGC | TGGGTGTGTCTACAGG | | |
| VEGF | ACTTCCCCAAATC | GTCACTCACTTTG | Seq. | 60° |
| G + 405C | ACTGTGG | CCCCTGT | | |
| VEGFR-2 | GCTTGTAGTAATTGTTCA | GAGCGTATGTCTACT | n.a. | 60° |
| (AC)ₙ repeat | TAAGTGG | ATACGCCA | | |
| Nrp1 | AGCTTTGGTTGGT | CCTGGAAACAAAA | Seq. | 60° |
| 3′UTR C/T | TTTGGTG | GGCATTC | | |
| AM | AAGAGGCTGAGTCAG | GCAACATCATTTTAATAT | n.a. | 60° |
| (CA)ₙ repeat | AAGGATTGG | CCTGCACAG | | |
| IL8 | TTGTTCTAACACCTG | GGCAAACCTGAGTC | Mfe I | 60° |
| T-251A | CCACTCT | TCACA | | |
| CXCR1 | CTCATGAGGACC | GGTTGAGGCAGCTA | Alu I | 60° |
| G + 2607C | CAGGTGAT | TGGAGA | | |
| CXCR2 | CATCTTTGCTGTCG | CTGTGAAGGATGCC | Seq. | 60° |
| C + 785T | TCCTCA | CAGAAT | | |
| EGF | CATTTGCAAACAG | TGTGACAGAGCAA | Alu I | 60° |
| A + 61G | AGGCTCA | GGCAAAG | | |
| EGFR | TGCTGTGACCCACT | CCAGAAGGTTGCACT | Bst-NI | 59° |
| G + 497A | CTGTCT | TGTCC | | |
| EGFR | ACCCCAGGGCTC | TGAGGGCACAAGAAG | n.a. | 55° |
| (CA)ₙ repeat | TATGGGAA | CCCCT | | |
| HIF-1α | CCCAATGGATGAT | AGTGGTGGCATTAGC | Tsp-45 I | 60° |
| C + 1772T | GACTTCC | AGTAGG | | |
| ARNT | ACAGGCAGGGTG | CACCTGTCAGGG | Seq. | 60° |
| PAS G/C | GTGTATGT | CATTTTCT | | |
| COX2 | GTTTGAAATTTTAA | TTTCAAATTATTGTT | BcII | 53° |
| T + 8473C | AGTACTTTTGAT | TCATTGC | | |

Statistical analysis: The primary endpoint in this study was time to tumor recurrence (TTR) in stage III colon cancer patients, which was defined as the time from the date of diagnosis of stage III colon cancer to the date of first recurrence, death, or until last contact if the patient was free of any tumor recurrence at the time of last contact. If a patient had not recurred, then TTR was censored at the time of death or at the last follow-up. The associations of time to tumor recurrence with patient's clinico-pathologic characteristics (age, sex, race, tumor grade, T-stage, N-stage and type of chemotherapy) were assessed using univariate survival analyses (log-rank test).

The adrenomedullin (CA). repeat (a/k/a AM 3′ UTR CA Repeat in Table 1) was analyzed by categorizing the patients into three groups: 1) patients carrying both alleles <14 repeats, 2) patients carrying one allele <14 and 3) patients carrying both alleles ≧14 repeats. The EGFR intron 1 (CA)_{n 16-23} repeat (a/k/a as EGFR Intron 1 at position 496 in Table 1) in each allele was categorized at the sample median, 20 (CA)ₙ, which was used in previous studies (Zhang et al. (2005) Clin. Cancer Res. 11:600-5 and Zhang et al. (2005) Clin. Colorectal Cancer 5:124-31). Other polymorphisms were coded according to their genotypes.

The association between each polymorphism and time to recurrence was examined using Kaplan-Meier curves and log-rank test. The distributions of polymorphisms across demographic characteristics were examined using Fisher's exact test. In the univariate survival analysis, the Pike estimate of relative risk (RR) and its associated 95% confidence interval (95% CI) was based on the log-rank test.

The Cox proportional hazards regression model with stratification factors (race, number of resected lymph nodes, and type of adjuvant therapy) was fitted to re-evaluate the association between polymorphisms and time to recurrence considering the imbalances in the distributions of baseline characteristics. P values of the log-likelihood ratio test were obtained from the modeling. Interactions between polymorphisms and gender, race, and type of adjuvant therapy on time to recurrence were tested by comparing corresponding likelihood ratio statistics between the baseline and nested Cox proportional hazards models that included the multiplicative product terms (Rothman (1998) MODERN EPIDEMIOLOGY Lippincott-Raven).

An internal validation analysis using bootstrapping was performed to reduce the possibility of overfitting or biased conclusions (Harrell et al. (1996) Stat. Med. 15:361-87). One thousand bootstrap samples were generated from the original sample. Each bootstrap sample consisted of 125 observations drawn from the original data set using simple random sampling with replacement (Chen et al. (1985) Stat. Med. 4:39-46). Variables chosen in the original analysis retained in multivariable analysis if associated P<0.05 in >50% of sample simulations.

All statistical tests were two-sided. Analyses were performed using the SAS statistical package version 9.1 (SAS Institute Inc., Cary, N.C., USA).

Results: A total of 125 patients with stage III colon cancer were included in this analysis: 50 women (40%) and 75 men (60%) with a median age of 58 years (range: 31-87 years). There were 70 Caucasian (56%), 22 Hispanic (18%), 26 Asian (21%), and 7 African-American (6%) study participants. All patients were diagnosed with stage III colon cancer during the years of 1992 and 2007. The median follow-up was 4.2 years.

Fifty-nine out of 125 patients had tumor recurrence, with a probability of 3-year recurrence of 0.45±0.047. The median time to recurrence was 5.2 years (95% Cl: 2.5-11.1 years). Fifty-one out of 59 (86%) patients showed recurrent disease within the first 3 years after surgery. Twenty-one patients showed one site of recurrence (36%), 22 patients (37%) displayed two sites of recurrence and 16 patients (27%) had 3 or more sites of recurrence. Thirty out of 59 patients (51%) recurred in the liver, 46% (27/59) recurred in the lung, 47% (28/59) showed peritoneal carcinomatosis and 36% (21/59) recurred in other organs. Thirty-six out of 125 patients have died and the median overall survival (OS) for the cohort is 11.9 years (95% CI: 5.8 to 14.3+). Patients with fewer than 12 lymph nodes removed were more likely to develop tumor recurrence (Median TTR of 2.5 years; CI: 1.4 to 6.6), compared to patients with more than 12 lymph nodes removed (median TTR: 7.1 years; CI: 2.8 to 10.4+) (log rang-test p=0.045). No significant associations between other demographic and clinico-pathologic variables and time to tumor recurrence were observed. Polymorphisms of VEGF (936 C/T, also shown as +936 C/T) and IL-8 were not associated with demographic (age, gender and ethnicity), clinical (type of chemotherapy), or pathologic characteristics (tumor grade and N-stage (N1/N2)).

VEGF C+936T and Time to Tumor Response (TTR) in Stage III Disease

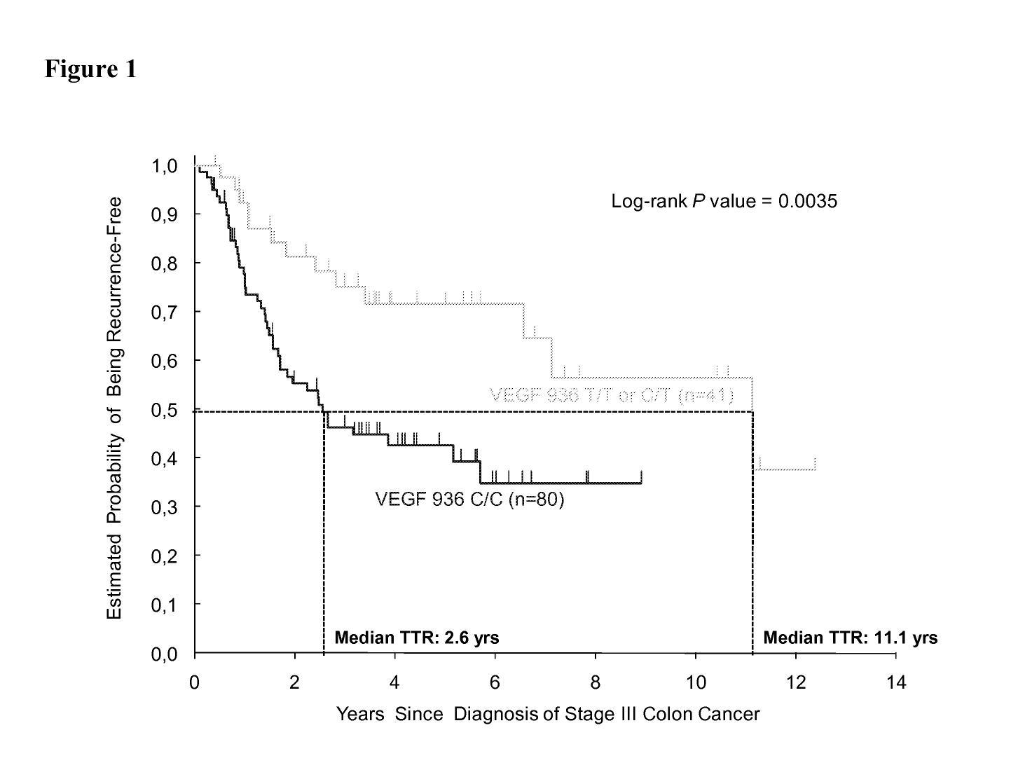

Sixty-six percent (80/121) of patients were homozygous for VEGF +936 C allele, 31% (37/121) were heterozygous (C/T), and 3% (4/121) were homozygous for the 936 T allele. The VEGF C+936T polymorphism showed significant association with time to tumor recurrence. Patients with the VEGF +936 C/C homozygous genotype had a median TTR of 2.6 years (95% CI: 1.7 to 5.7 years), compared to 11.1 years (95% CI: 6.6 to 12.4+years) in patients heterozygous or homozygous for the T-allele (p=0.003, log-rank test, FIG. 1).

IL-8 T-251A and Time to Tumor Response (TTR) in Stage III Disease

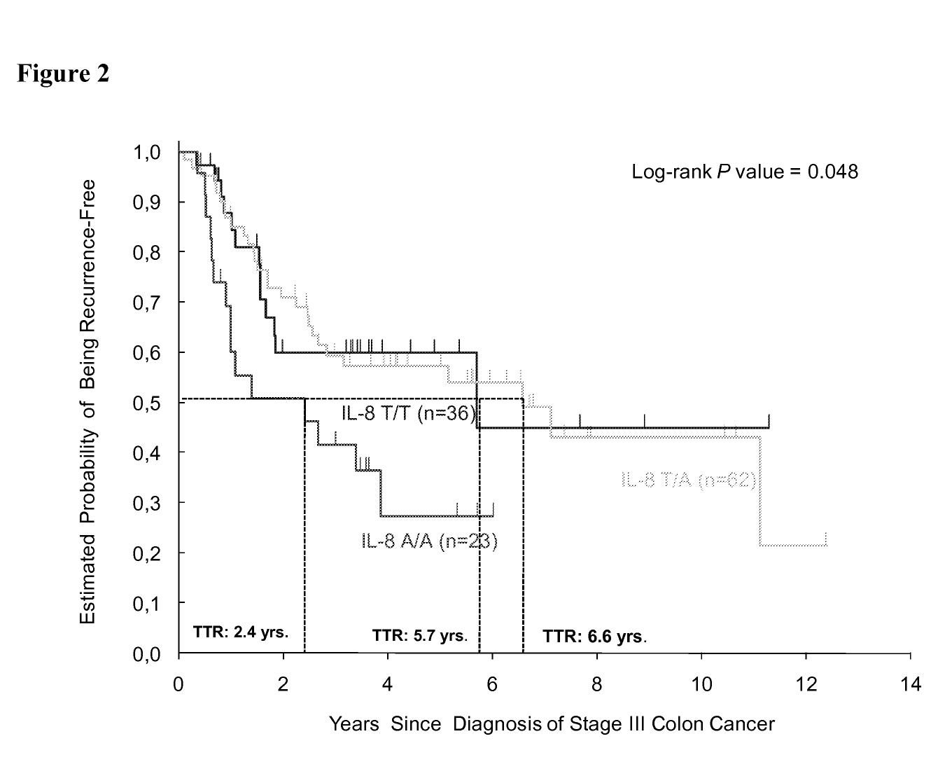

Thirty percent (36/121) of patients were homozygous for the IL-8 −251 T-allele, 51% (62/121) were heterozygous (T/A), and 19% (23/121) were homozygous for the −251 A allele. The IL-8 T-251A polymorphism showed a significant association with time to tumor recurrence. Patients with the IL-8 −251 A/A homozygous genotype had a median time to recurrence of 2.4 years (95% CI: 1.0 to 3.9 years), compared to 6.6 years (95% CI: 2.7 to 12.4+ years) for those with heterozygous −251 T/A allele and 5.7 years for homozygous −251 T-allele carriers (95% CI: 1.8 to 11.3+ years) (p=0.048, log-rank test, FIG. 2).

Multivariable Analysis of IL-8 T-251A and VEGF C+936T

When IL-8 T-251A (adjusted p-value=0.030) and VEGF C+936T (adjusted p-value<0.001) were analyzed jointly, stratified by race, number of resected lymph nodes and type of adjuvant therapy, the two polymorphisms remained significantly associated with time to recurrence (Table 7). Bootstrap analysis confirmed that polymorphisms were selected for the final multivariable model in 88% for VEGF C+9361 and 56% for IL-8 of the 1000 bootstrap samples as predictive factors significantly associated with time to recurrence at the 0.05 level.

TABLE 7
Multivariable analysis of VEGF and IL-8 polymorphisms and time to
recurrence

| | | Adjusted RR | |
|---|---|---|---|
| | n | (95% CI)* | Adjusted P value |
| VEGF C + 936T | | | |
| C/C (unfavorable) | 80 (66.1%) | 1 (Reference) | <0.001 |
| C/T, T/T (favorable) | 41 (33.9%) | 0.28 (0.12, 0.64) | |
| IL 8 T-251A | | | |
| T/T, T/A (favorable) | 98 (81%) | 1 (Reference) | 0.030 |
| A/A (unfavorable) | 23 (19%) | 2.24 (1.12, 4.47) | |
| Combined† | | | |
| 2 favorable | 33 (27.3%) | 1 (Reference) | <0.001 |
| 1 favorable | 73 (60.3%) | 5.04 (1.69, 15.0) | |
| 0 favorable | 15 (12.4%) | 9.45 (2.74, 32.6) | |

*Based on Cox proportional hazards model, stratified by race, number of resected lymph nodes, and type of adjuvant therapy, with 2 polymorphisms included.
†Based on Cox proportional hazards model, stratified by race, number of resected lymph nodes, and type of adjuvant therapy.

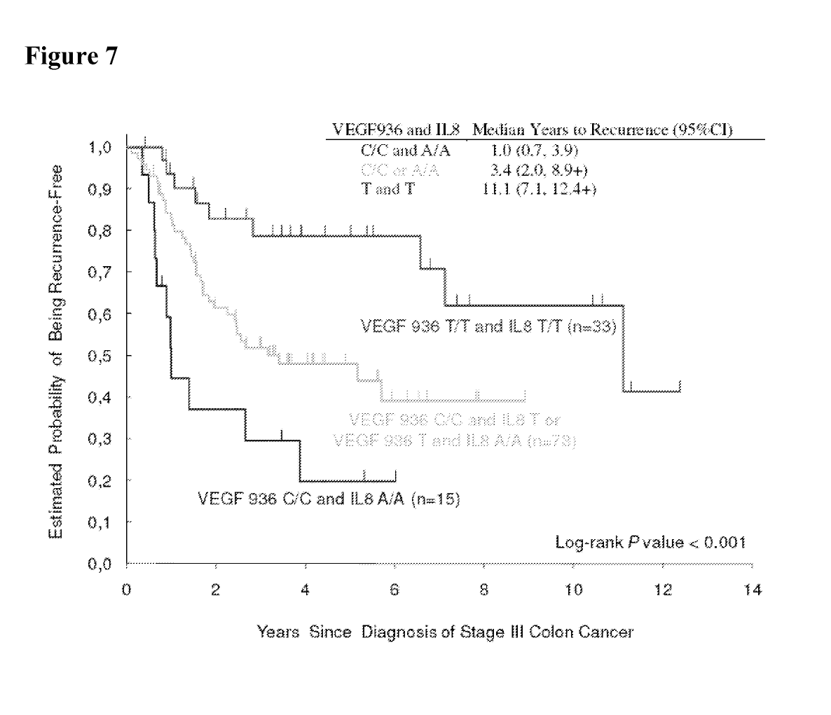

In a combined analysis, there was a statistically significant relationship between the two polymorphisms and time to tumor recurrence. Patients with VEGF +936 C/C and IL-8 −251 A/A genotype were at greatest risk to develop tumor recurrence (TTR=1.0 year, CI: 0.7-3.9), compared to patients displaying the combination of VEGF +936 T/T and IL-8 −251 T/T genotype, who were less likely to develop tumor recurrence (TTR=11.1 years, CI: 7.1-12.4+) (p<0.001, log-rank test; FIG. 7).

Analysis of Interactions Between IL-8 T-251A and VEGF C+936T and Sex, Race, and Type of Adjuvant Therapy on Time to Recurrence

The associations between IL-8 T-251A and VEGF C+936T and time to recurrence differed by sex, race, and type of adjuvant therapy was tested and no significant interactions were found.

Analysis of Other Tested Germline Polymorphisms Involved in the Tumor Angiogenesis Pathway

No statistically significant associations between other tested genes involved in tumor angiogenesis pathway (n=12) and time to tumor recurrence (Table 4) were observed.

Experimental Discussion:

Like normal tissues, tumors require an adequate supply of oxygen, metabolites and an effective way to remove waste products. Therefore, gaining access to the host vascular system and the generation of a tumor blood supply are rate-limiting steps in tumor growth and progression (Bergers et al. (2003) Nat. Rev. Cancer 3:401-10). Tumors start as avascular masses which can initially thrive on pre-existent vasculature within the microenvironment (van Kempen et al. (2006) Eur. J. Cell Biol. 85:61-8). When a tumor grows beyond a size of approximately 2-3 mm, as a consequence, the tumor requires its own new and dedicated vasculature. The so called “angiogenic switch”, the induction of tumor vasculature or switch to an angiogenic phenotype, is considered a hallmark of the malignant process and is required for tumor propagation and progression (de Castro et al. (2006) Crit. Rev. Oncol. Hematol. 59:40-50 and Hicklin et al. (2005) J. Clin. Oncol. 23:1011-27). In contrast to physiological angiogenesis, solid tumors lose the appropriate balance between positive and negative control by inducing mainly proangiogenic factors (Bergers et al. (2003) Nat. Rev. Cancer 3:401-10 and Carmeliet P. (2005) Nature 438:932-6).

Vascular endothelial growth factor is one of the most important activators of tumor associated angiogenesis (Hicklin et al. (2005) J. Clin. Oncol. 23:1011-27). Activation of the VEGF/VEGF-receptor axis triggers multiple signaling pathways that result in endothelial cell survival, mitogenesis, migration, differentiation, vascular permeability and mobilization of endothelial progenitor cells (Dvorak HF (2002) J. Clin. Oncol. 20:4368-80). Overexpression of VEGF mRNA and protein has been associated with tumor progression and poor prognosis in a variety of malignancies, including melanoma, ovarian carcinoma, prostate carcinoma and colon carcinoma (Decaussin et al. (1999) J. Pathol. 188:369-77; Ferrer et al. (1999) Urology 54:567-72; Masood et al. (2001) Blood 98:1904-13 and Takahashi et al. (1997) Arch. Surg. 132:541-6). Its expression level in cancer cells directly correlates with tumor size, metastasis and poor prognosis in many types of solid and hematological tumors (Dvorak H F (2002) J. Clin. Oncol. 20:4368-80 and Ribatti et al. (2005) Peptides 26:1 670-5). DNA sequence variations within the VEGF gene lead to altered VEGF production and/or activity. Several polymorphisms within the VEGF gene have been described. A C to T change at position 936 within the 3′-UTR region of the VEGF gene has been associated with decreased plasma levels of VEGF, as shown by Renner and coworkers (Renner et al. (2000) J. Vasc. Res. 37:443-8). Numerous studies reported on associations between VEGF C+936T polymorphism and susceptibility to cancer and other diseases, including breast- and lung cancer (Krippl et al. (2003) Int. J Cancer 106:468-71; Lee et al. (2005) Cancer Epidemiol Biomarkers Prey. 14:571-5; Eroglu et al. (2006) Ann. Oncol. 17:1467-8; Medford et al. (2005) Thorax 60:244-8 and Nam et al. (2005) Hum. Immunol. 66:1068-73). A recent study demonstrated that the prevalence of the VEGF +936T allele, which is associated with decreased plasma levels of VEGF, was less common in breast cancer patients than in healthy subjects, indicating that this genetic variant may be protective against breast cancer (Krippl et al. (2003) Int. J. Cancer 106:468-71). To date, angiogenesis related germline polymorphisms have not been reported to be causatively linked to time to tumor recurrence or clinical outcomes in adjuvant colon cancer patients. In this study, “high-expression” variants of VEGF C+936T (VEGF +936 C/C) were found to be significantly associated with time to tumor recurrence in both univariate and multivariable analysis (Table 5, Table 7, FIG. 1). These findings demonstrate for the first time that VEGF C+936T may be an important prognostic factor for stage III colon cancer, indicating a potential role of tumor associated angiogenesis in the development of colon cancer tumor relapse.

Recently interleukin (IL-8), a member of the CXC chemokine family, has been found to be a critical VEGF-independent mediator of tumor associated angiogenesis (Strider et al. (2006) Eur. J. Cancer 42:768-78). IL-8 exerts its potent angiogenic properties on endothelial cells through interaction with its receptors CXCR1 and CXCR2 (Li et al. (2003) J. Immunol. 170:3369-76 and Venkatakrishnan et al. (2000) J. Biol. Chem. 275:6868-75). Induction of IL-8 preserved the angiogenic phenotype in HIF1-α deficient colon cancer cells, suggesting a critical role of IL-8 in tumor associated angiogenesis, independently of VEGF (Strieter et al. (2006) Eur. J. Cancer 42:768-78 and Strieter R M (2005) Nat. Med. 11:925-7). Overexpression of IL-8 has been found to be associated with VEGF independent angiogenesis, advanced disease stage, lymphovascular invasion, poor prognosis and tumor recurrence in several different malignancies, including non-small cell lung cancer and rectal cancer (Strieter et al. (2006) Eur. J. Cancer 42:768-78; Strieter R M (2005) Nat. Med. 11:925-7; Gordon et al. (2006) Pharmacogenomics 7:67-88 and Yuan et al. (2000) Am. J. Respir. Crit. Care Med. 162:1957-63). Additionally, colorectal cancer patients with lung- and liver metastases, have been found to have elevated plasma levels of IL-8 (Ueda et al. (1994) J. Gastroenterol. 29:423-9), indicating a potential role in VEGF independent angiogenesis and tumor metastases. Hull et al. identified a common single nucleotide polymorphism 251 base pairs upstream of the IL-8 transcription start site. In their in vivo models, they demonstrated, that the IL-8 −251A allele was associated with increased plasma levels of IL-8 (Hull et al. (2000) Thorax 55:1023-7). As previously demonstrated, IL-8 T-251A polymorphism and its receptor CXCR-1 were associated with clinical outcome in colon and rectal cancer patients (Zhang et al. (2005) Clin. Colorectal Cancer 5:124-31; Gordon et al. (2006) Pharmacogenomics 7:67-88). IL-8 T-251A polymorphism was found to be significantly associated with risk of recurrence in rectal cancer patients in both univariate and regression tree analysis (Gordon et al. (2006) Pharmacogenomics 7:67-88). The present study shows, that stage III colon cancer patients harboring high expression variants of IL-8 T-251A polymorphism (A/A genotype) were at higher risk of developing tumor recurrence (FIG. 2), supporting the hypothesis that increased angiogenic potential is critical for tumor relapse.

A combined analysis of VEGF C+936T and IL-8 T-251A showed a statistically significant relationship between the two polymorphisms and time to tumor recurrence. Grouping alleles into favorable vs. non-favorable alleles, “high expression” variants of VEGF C+936T and IL-8 T-251A (VEGF +936 C and IL-8 -251 A) were associated with a higher likelihood of developing tumor recurrence (Table 7) (p<0.001, log-rank test). In addition, multivariate analysis confirmed that VEGF C+936T (adjusted p-value<0.001) and IL-8 T-251A (adjusted p-value=0.030) were significantly associated with time to tumor recurrence (FIG. 7).

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for determining whether a colon cancer patient will likely respond to a therapy comprising the administration of an effective amount of a pyrimidine based antimetabolite and an effective amount of an efficacy enhancing agent comprising screening a suitable cell or tissue sample isolated from said patient for at least one genetic polymorphism selected from:
(i) VEGF at nt 936 C/T;
(ii) IL-8 at nt −251 T/A;
(iii) VEGFR2 (KDR) at position 4422;
(iv) the number of 20 CA repeats for EGFR at 496 in Intron I;
(v) the number of alleles with <14 CA repeats for AM 3′UTR CA repeat;
(vi) IL-1β at nt 3954 C/T, or
(vii) VEGF at nt 936 C/T and IL-8 at nt −251 T/A;
wherein the presence of at least one of the following said respective genetic polymorphism identifies the patient as likely to respond to said therapy:
(viii) (C/T or T/T) for VEGF at nt 936 C/T;
(ix) (T/T or T/A) for IL-8 at nt −251 T/A;
(x) (11/12 or 11/11 AC repeats) for VEGFR2 (KDR) at position 4422;
(xi) (2 alleles having <20 CA repeats) for EGFR at 496 CA repeats in Intron I;
(xii) (2 alleles with <14 CA repeats or 2 alleles with ≧14 CA repeats) for AM 3′UTR CA repeat;
(xiii) (C/C or C/T) for IL-1β at nt 3954 C/T;
(xiv) (T/T) for VEGF at nt 936 C/T and (T/T) for IL-8 at nt −251 T/A;
(xv) (C/C) for VEGF at nt 936 C/T and (T/T or T/A) for IL-8 at nt −251 T/A, or
(xvi) (C/T or T/T) for VEGF at nt 936 C/T and (A/A) for IL-8 −251 T/A.

2. The method of claim 1, wherein the chemotherapy further comprises the administration of an effective amount of a platinum-based alkylating agent.

3. The method of claim 1 or 2, wherein the chemotherapy further comprises the administration of an effective amount of a topoisomerase I inhibitor.

4. The method of claim 1, wherein the pyrimidine based antimetabolic is 5-Fluoruracil or an equivalent thereof.

5. The method of claim 1, wherein the efficacy enhancing agent is Leucovorin or an equivalent thereof.

6. The method of claim 1, wherein the patient is diagnosed with stage II or stage III colon cancer.

7. The method of claim 1, wherein the patient is diagnosed with stage II colon cancer and wherein the presence of at least one of the following said respective genetic polymorphism identifies the patient as likely to respond to said therapy:
(i) (11/12 or 11/11 AC repeats) for VEGFR2 (KDR) at position 4422;
(ii) (2 alleles having <20 CA repeats) for EGFR at 496 CA repeats in Intron I;
(iii) (2 alleles with <14 CA repeats or 2 alleles with ≧14 CA repeats) for AM 3′UTR CA repeat; or
(iv) (C/C or C/T) IL-1β at nt 3954 C/T.

8. The method of claim 1, wherein the patient is diagnosed with stage III colon cancer and wherein the presence of at least one of the following said respective genetic polymorphism identifies the patient as likely to respond to said therapy:
(i) (C/T or T/T) for VEGF at nt 936 C/T;
(ii) (T/T or T/A) for IL-8 at nt −251 T/A;
(iii) (T/T) for VEGF at nt 936 C/T and (T/T) for IL-8 at nt −251 T/A;
(iv) (C/C) for VEGF at nt 936 C/T and (T/T or T/A) for IL-8 at nt −251 T/A; or
(v) (C/T or T/T) for VEGF at nt 936 C/T and (A/A) for IL-8 -251 T/A.

9. The method of claim 1, wherein the suitable cell or tissue sample comprises a tumor cell or tissue sample.

10. The method of claim 1, wherein the suitable cell or tissue sample comprises peripheral blood lymphocytes.

11. The method of claim 1, wherein the likelihood of response to said therapy is a delay in the time to tumor recurrence in said patient.

12. A method for treating a gastrointestinal or lung cancer patient identified by:
(a) having a genetic polymorphism selected from the group consisting of (C/T or T/T) for VEGF at nt 936 C/T; (T/T or T/A) for IL-8 at nt −251 T/A; (11/12 or 11/11 AC repeats) for VEGFR2 (KDR) at position 4422; (2 alleles having <20 CA repeats) for EGFR at 496 CA repeats in Intron I; (2 alleles with <14 CA repeats or 2 alleles with ≧14 CA repeats) for AM 3′UTR CA repeat; (C/C or C/T) for IL-1β at nt 3954 C/T; (T/T) for VEGF at nt 936 C/T and (T/T) for IL-8 at nt −251 T/A; (C/C) for VEGF at nt 936 C/T and (T/T or T/A) for IL-8 at nt −251 T/A; or (C/T or T/T) for VEGF at nt 936 C/T and (A/A) for IL-8 −251 T/A, in a suitable cell or tissue sample isolated from said patient, and then
(b) administering an effective amount of a pyrimidine based antimetabolite and an efficacy enhancing agent based chemotherapy to the patient identified in step (a), thereby treating the patient.

13. The method of claim 12, wherein the chemotherapy further comprises the administration of an effective amount of a platinum-based alkylating agent.

14. The method of claim 12 or 13, wherein the chemotherapy further comprises the administration of an effective amount of a topoisomerase I inhibitor.

15. The method of claim 12, wherein the pyrimidine based antimetabolie is 5-Fluoruracil or an equivalent thereof.

16. The method of claim 12, wherein the efficacy enhancing agent is Leucovorin or an equivalent thereof.

17. The method of claim 12, wherein the patient is diagnosed with stage II or stage III colon cancer.

18. The method of claim 12, wherein the patient is diagnosed with stage II colon cancer and wherein the patient has at least one of the following said respective genetic polymorphisms identified in step (a):
(i) (11/12 or 11/11 AC repeats) for VEGFR2 (KDR) at position 4422;
(ii) (2 alleles having <20 CA repeats) for EGFR at 496 CA repeats in Intron I;
(iii) (2 alleles with <14 CA repeats or 2 alleles with ≧14 CA repeats) for AM 3′UTR CA repeat; or
(iv) (C/C or C/T) IL-1β at nt 3954 C/T.

19. The method of claim 12, wherein the patient is diagnosed with stage III colon cancer and wherein the patient has at least one of the following said respective genetic polymorphisms identified in step (a):
(i) (C/T or T/T) for VEGF at nt 936 C/T;
(ii) (T/T or T/A) for IL-8 at nt −251 T/A;
(iii) (ITT) for VEGF at nt 936 C/T and (T/T) for IL-8 at nt −251 T/A;
(iv) (C/C) for VEGF at nt 936 C/T and (T/T or T/A) for IL-8 at nt −251 T/A; or
(v) (C/T or T/T) for VEGF at nt 936 C/T and (A/A) for IL-8 −251 T/A.

20. The method of claim 12, wherein the suitable cell or tissue sample comprises a tumor cell or tissue sample.

21. The method of claim 12, wherein the suitable cell or tissue sample comprises peripheral blood lymphocytes.

22. A method for selecting a chemotherapy for a colon cancer patient in need of additional therapy or is less likely to benefit from pyrimidine based antimetabolite and efficacy enhancing agent based chemotherapy, comprising screening a suitable cell or tissue sample isolated from said patient for at least one genetic polymorphism selected from:
(i) (C/C) for VEGF at nt 936 C/T;
(ii) (A/A) for IL-8 at nt −251 T/A;
(iii) (12/12 AC repeats) for VEGFR2 (KDR) at position 4422;
(iv) (at least one allele with ≧20 CA repeats) for EGFR at 496 CA repeats in Intron I;
(v) VEGF at nt 936 C/T and (A/A) for IL-8 at nt −251 T/A.
wherein the presence of at least one genetic polymorphism selects the patient as in need of additional therapy or is less likely to respond to said chemotherapy.

23. The method of claim 22, wherein the chemotherapy further comprises the administration of an effective amount of a platinum-based alkylating agent.

24. The method of claim 22 or 23, wherein the chemotherapy further comprises the administration of an effective amount of a topoisomerase I inhibitor.

25. The method of claim 22, wherein the pyrimidine based antimetabolie is 5-Fluoruracil or an equivalent thereof.

26. The method of claim 22, wherein the efficacy enhancing agent is Leucovorin or an equivalent thereof.

27. The method of claim 22, wherein the patient is diagnosed with stage II or stage III colon cancer.

28. The method of claim 22, wherein the patient is diagnosed with stage II colon cancer and wherein the presence of at least one of the following said respective genetic polymorphism identifies the patient as in need of additional chemotherapy or less likely to respond to said therapy:
(i) (12/12 AC repeats) for VEGFR2 (KDR) at position 4422;
(ii) (at least one allele with ≧20 CA repeats) for EGFR at 496 CA repeats in Intron I;
(iii) (only 1 allele with ≧14 CA repeats) for AM 3′UTR CA repeat; or
(iv) (T/T) IL-1β at nt 3954 C/T.

29. The method of claim 22, wherein the patient is diagnosed with stage III colon cancer and wherein the presence of at least one of the following said respective genetic polymorphism identifies the patient as in need of additional chemotherapy or less likely to respond to said therapy:
(i) (C/C) for VEGF at nt 936 C/T;
(ii) (A/A) for IL-8 at nt −251 T/A;
(iii) (C/C) for VEGF at nt 936 C/T and (A/A) for IL-8 at nt −251 T/A;
(iv) (C/C) for VEGF at nt 936 C/T and (T/T or T/A) for IL-8 at nt −251 T/A; or
(v) (C/T or VT) for VEGF at nt 936 C/T and (A/A) for IL-8 −251 T/A.

30. The method of claim 22, wherein the suitable cell or tissue sample comprises a tumor cell or tissue sample.

31. The method of claim 22, wherein the suitable cell or tissue sample comprises peripheral blood lymphocytes.

32. The method of claim 22, wherein the likelihood of response to said therapy is a delay in the time to tumor recurrence in said patient.

33. A prognostic panel of genetic markers comprising a primer or nucleic acid probe that identifies the genotype of a patient sample for at least one or more genetic polymorphism of the group:
(i) VEGF at nt 936 C/T;
(ii) IL-8 at nt −251 T/A;
(iii) VEGFR2 (KDR) at position 4422;
(iv) the number of 20 CA repeats for EGFR at 496 in Intron I;
(v) the number of alleles with <14 CA repeats for AM 3′UTR CA repeat;
(vi) IL-1β at nt 3954 C/T; or
(vii) VEGF at nt 936 C/T and IL-8 at nt −251 T/A.

34. The panel of claim 33, wherein the probes or primers are attached to a microarray.

35. The panel of claim 33, wherein the probes or primers are detectably labeled.

36. The panel of claim 33, wherein the probes or primers identify the genotype of a plurality of polymorphisms selected from the group consisting of: at least two, at least three, at least four, at least five, at least six and all seven of the genetic polymorphisms.

37. The panel of claim 33, wherein the panel determines whether a colon cancer patient in need thereof will likely respond to a therapy comprising the administration of an effective amount of a pyrimidine based antimetabolite and an effective amount of an efficacy enhancing agent.

38. The panel of claim 33, wherein the panel determines whether a colon cancer patient in need of additional therapy is most likely to benefit from pyrimidine based antimetabolite and efficacy enhancing agent based chemotherapy.

39. The panel of claim 37 or 38, wherein the pyrimidine based antimetabolite and efficacy enhancing agent comprises 5-FU adjuvant therapy.

40. The panel of claim 39, wherein the 5-FU adjuvant therapy comprise 5-FU or an equivalent thereof and Leucovorin or an equivalent thereof.

41. The panel of claim 38, wherein the therapy further comprises the administration of an effective amount of a toposiomerase I inhibitor.

42. The panel of claim 38 or 41, wherein the therapy further comprises the administration of an effective amount of a platinum-based alkylating agent.
